SEQUENCE LISTING (34 sequences)

SEQ ID NO: 1 - DNA, 1650 bp, Bacillus stearothermophilus
gtgctaacgtttcaccgcatcattcgaaaaggatggatgttcctgctcgcgtttttgctcactgcctcgctgttctgcccaacaggacagcacgccaaggctgccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgactgggacgaaagccgaaaattaagccgcatttacaaattccgcggcatcggcaaagcgtgggattgggaagtagacacggaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgctgcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgaccgtttctaccatcgctcggccgatcacaacccgaccgtggactggtgaattcgtccgttggaccgaaccacggttggtggcatggccttga

SEQ ID NO: 2 - protein, 546 aa, Bacillus stearothermophilus
MLTFHRIIRKGWMFLLAFLLTASLFCPTGRHAKAAAPFNGTMMQYFEWYLPDDGTLWTKVANEANNLSSLGITALSLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFIGKAWDWEVDTENGNYDYLMYADLDMDHPEVVTELKNWGKWYVNTTNIDGFRLDGLKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTNPAKRCSHGRPWFKPLAYAFILTRQEGYPCVFYGDYYGIPQYNIPSLKSKIDPLLIARRDYAYGTQHDYLDHSDIIGWTREGVTEKPGSGLAALITDGAGRSKWMYVGKQHAGKVFYDLTGNRSDTVTINSDGWGEFKVNGGSVSVWVPRKTTVSTIARPITTRPWTGEFVRWHEPRLVAWP

SEQ ID NO: 3 - protein, 515 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 4 - protein, 514 aa, Artificial Sequence
  recombinantly produced variant
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGRADGTEWVDAVEVNPSDRNQEISGTYQIQAWTKFDFPGRGNTYSSFKWRWYHFDGVDWDESRKLSRIYKFIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 5 - protein, 512 aa, Bacillus licheniformis
MKQQKRLYARLLTLLFALIFLLPHSAAAAANLNGTLMQYFEWYMPNDGQHWKRLQNDSAYLAEHGITAVWIPPAYKGTSQADVGYGAYDLYDLGEFHQKGTVRTKYGTKGELQSAIKSLHSRDINVYGDVVINHKGGADATEDVTAVEVDPADRNRVISGEHRIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNSTVVSKHPLKAVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 6 - protein, 514 aa, Bacillus amyloliquefaciens
MIQKRKRTVSFRLVLMCTLLFVSLPITKTSAVNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 7 - protein, 549 aa, Bacillus stearothermophilus
MLTFHRIIRKGWVFLLAFWLTASLFCPTGQPAKAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFNGRGNTYSSFKWRWYHFDGVDWDESRKLSRIYKFRGIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMSTLMNNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWPITTRPWTGEFVRWTEPRLVAWP

SEQ ID NO: 8 - DNA, 1626 bp, Artificial Sequence
  synthetic nucleotide encoding fusion protein
atgaaacaacaaaaacggctttacgcccgattgctgacgctgttatttgcgctcatcttcttgctgcctcattctgcagcttcagcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcaccataaaggcggcgctgaccacgccgctggaatgcaagtgtacgccgatgtcgtgttctttcaagccattcaagccgccggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgactgggacgaaagccgaaaattaagccgcatttacaaattcatcggcaaagcgtgggattgggaagtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgctgcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgaccgtttctaccatcgctcggccgatcacaacccgaccgtggactggtgaattcgtccgttggaccgaaccacggttggtggcatggcct

SEQ ID NO: 9 - protein, 542 aa, Artificial Sequence
  synthetic fusion protein
MKQQKRLYARLLTLLFALIFLLPHSAASAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 10 - protein, 4 aa, Bacillus stearothermophilus
DINK

SEQ ID NO: 11 - protein, 4 aa, Bacillus stearothermophilus
FIGK

SEQ ID NO: 12 - protein, 4 aa, Bacillus stearothermophilus
GAFD

SEQ ID NO: 13 - protein, 5 aa, Bacillus stearothermophilus
EGGTE

SEQ ID NO: 14 - protein, 4 aa, Bacillus stearothermophilus
ARPI

SEQ ID NO: 15 - protein, 4 aa, Bacillus stearothermophilus
TGEF

SEQ ID NO: 16 - protein, 484 aa, Artificial Sequence
  recombinantly produced alpha amylase
AAPFNGTMMQYFEWYLPDDGTLWTKVANEANNLSSLGITALWLPPATKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTT

SEQ ID NO: 17 - protein, 29 aa, Bacillus stearothermophilus
VSTIARPITTRPWTGEFVRWTEPRLVAWP

SEQ ID NO: 18 - DNA, 44 bp, Artificial Sequence
  primer
gaatgtctgcagcttcagcagccgcaccgtttaacggcaccatg

SEQ ID NO: 19 - DNA, 36 bp, Artificial Sequence
  primer
cccggggttaactcaaggccatgccaccaaccgtgg

SEQ ID NO: 20 - DNA, 30 bp, Artificial Sequence
  primer
cccggccaagcgcttcagtcatgggtcgac

SEQ ID NO: 21 - DNA, 30 bp, Artificial Sequence
  primer
gtcgacccatgactgaagcgcttggccggg

SEQ ID NO: 22 - DNA, 39 bp, Artificial Sequence
  primer
atcctactcgaggcttttcttttggaagaaaatataggg

SEQ ID NO: 23 - DNA, 35 bp, Artificial Sequence
  primer
tggaatctcgaggttttatcctttaccttgtctcc

SEQ ID NO: 24 - DNA, 44 bp, Artificial Sequence; identical to SEQ ID NO: 18
  primer

SEQ ID NO: 25 - DNA, 36 bp, Artificial Sequence; identical to SEQ ID NO: 19
  primer

SEQ ID NO: 26 - DNA, 30 bp, Artificial Sequence; identical to SEQ ID NO: 20
  primer

SEQ ID NO: 27 - DNA, 30 bp, Artificial Sequence; identical to SEQ ID NO: 21
  primer

SEQ ID NO: 28 - DNA, 39 bp, Artificial Sequence; identical to SEQ ID NO: 22
  primer

SEQ ID NO: 29 - DNA, 35 bp, Artificial Sequence; identical to SEQ ID NO: 23
  primer

SEQ ID NO: 30 - protein, 547 aa, Artificial Sequence
  recombinantly produced variant alpha amylase
MLTFHRIIRKGWMFLLAFLLTASLFCPTGQHAKAAAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFIGKAWDWEVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIARPITTRPWTGEFVRWTEPRLVAWP

SEQ ID NO: 31 - protein, 4 aa, Bacillus stearothermophilus
SGGA

SEQ ID NO: 32 - protein, 5 aa, Bacillus stearothermophilus
TREGV

SEQ ID NO: 33 - protein, 4 aa, Bacillus stearothermophilus
TITR

SEQ ID NO: 34 - protein, 4 aa, Bacillus stearothermophilus
PWTG

(19) United States
(12) Patent Application Publication
(10) Pub. No.: US 2006/0014265 A1
(43) Pub. Date: Jan. 19, 2006

(54) Mutant alpha-amylases

(76) Inventors: Eugenio Ferrari, Palo Alto, CA (US); Marc Kolkman, Ae Leiden (NL); Craig E. Pilgrim, Beloit, WI (US)

Correspondence Address: GENENCOR INTERNATIONAL, INC., 925 PAGE MILL ROAD, PALO ALTO, CA, 94304-1013, US

(21) Appl. No.: 11/102,188

(22) Filed: Apr. 8, 2005

Related U.S. Application Data
(60) Provisional application No. 60/561,124, filed on Apr. 8, 2004.

(51) Int. Cl. C11D 3/386; C07H 21/04; C12P 21/06; C12P 19/02; C12N 9/32; C12N 15/74

(52) U.S. Cl. 435/204; 435/69.1; 435/252.31; 435/471; 536/23.2; 435/105; 510/320

(57) ABSTRACT

Novel variant α-amylase enzymes are disclosed in which the residues corresponding to R179 and G180 in Bacillus stearothermophilus (SEQ ID NO.2) are deleted. The disclosed variant α-amylase enzymes show altered or improved stability and/or activity profiles.

### CROSS-REFERENCE TO RELATED APPLICATIONS

The present application claims priority to U.S. Provisional Patent Application Ser. No. 60/561,124, entitled “Mutant α-Amylases”, filed Apr. 8, 2004.

### FIELD OF THE INVENTION

The present invention is directed to α-amylases having introduced therein mutations providing altered performance characteristics, such as altered stability and/or altered activity profiles. Further, the invention also relates to truncated α-amylases.

### BACKGROUND OF THE INVENTION

α-Amylases (α-1,4-glucan-4-glucanohydrolase, EC 3.2.1.1) hydrolyze internal α-1,4-glucosidic linkages in starch, largely at random, to produce smaller molecular weight malto-dextrins. α-Amylases are of considerable commercial value, being used in the initial stages (liquefaction) of starch processing; in alcohol production; as cleaning agents in detergent matrices; and in the textile industry for starch desizing. α-Amylases are produced by a wide variety of microorganisms including Bacillus and Aspergillus, with most commercial amylases being produced from bacterial sources such as Bacillus licheniformis, Bacillus amyloliquefaciens, Bacillus subtilis, or Bacillus stearothermophilus. In recent years, the preferred enzymes in commercial use have been those from Bacillus licheniformis because of their heat stability and performance under commercial operating conditions.

In general, starch to fructose processing consists of four steps: liquefaction of granular starch, saccharification of the liquefied starch into dextrose, purification, and isomerization to fructose. The object of a starch liquefaction process is to convert a concentrated suspension of starch polymer granules into a solution of soluble shorter chain length dextrins of low viscosity. This step is essential for convenient handling with standard equipment and for efficient conversion to glucose or other sugars. To liquefy granular starch, it is necessary to gelatinize the granules by raising the temperature of the granular starch to over about 72° C. The heating process instantaneously disrupts the insoluble starch granules to produce a water soluble starch solution. The solubilized starch solution is then liquefied by α-amylase (EC 3.2.1.1.).

A common enzymatic liquefaction process involves adjusting the pH of a granular starch slurry to between 6.0 and 6.5, the pH optimum of α-amylase derived from Bacillus licheniformis, with the addition of calcium hydroxide, sodium hydroxide or sodium carbonate. The addition of calcium hydroxide has the advantage of also providing calcium ions which are known to stabilize the α-amylases against inactivation. Upon addition of α-amylases, the suspension is pumped through a steam jet to instantaneously raise the temperature to between 80-115° C. The starch is immediately gelatinized and, due to the presence of α-amylases, depolymerized through random hydrolysis of a (1-4) glycosidic bonds to a fluid mass which is easily pumped.

In a second variation to the liquefaction process, α-amylase is added to the starch suspension, the suspension is held at a temperature of 80-100° C. to partially hydrolyze the starch granules, and the partially hydrolyzed starch suspension is pumped through a jet at temperatures in excess of about 105° C. to thoroughly gelatinize any remaining granular structure. After cooling the gelatinized starch, a second addition of α-amylase can be made to further hydrolyze the starch.

A third variation of this process is called the dry milling process. In dry milling, whole grain is ground and combined with water and/or thin stillage. The germ is optionally removed by flotation separation or equivalent techniques. The resulting mixture, which contains starch, fiber, protein and other components of the grain, is liquefied using α-amylase. The general practice in the art is to undertake enzymatic liquefaction at a lower temperature when using the dry milling process. Generally, low temperature liquefaction is believed to be less efficient than high temperature liquefaction in converting starch to soluble dextrins.

Typically, after gelatinization the starch solution is held at an elevated temperature in the presence of α-amylase until a DE of 8-20 is achieved, usually a period of 1-3 hours. Dextrose equivalent (DE) is the industry standard for measuring the concentration of total reducing sugars, calculated as D-glucose on a dry weight basis. Unhydrolyzed granular starch has a DE of virtually zero, whereas the DE of D-glucose is defined as 100.

The maximum temperature at which the starch solution containing α-amylase can be held depends upon the microbial source from which the enzyme was obtained and the molecular structure of the α-amylase molecule. α-Amylases produced by wild type strains of Bacillus subtilis or Bacillus amyloliquefaciens are typically used at temperatures no greater than about 90° C. due to excessively rapid thermal inactivation above that temperature, whereas α-amylases produced by wild type strains of Bacillus licheniformis can be used at temperatures up to about 110° C. The presence of starch and calcium ion are known to stabilize α-amylases against inactivation.

Subsequent to liquefaction, the processed starch is saccharified to glucose with glucoamylase. A problem with present processes occurs when residual starch is present in the saccharification mixture due to an incomplete liquefaction of the starch, e.g., inefficient amylose hydrolysis by amylase. Residual starch is highly resistant to glucoamylase hydrolysis. It represents a yield loss and interferes with downstream filtration of the syrups.

Additionally, many α-amylases are known to require the addition of calcium ion for stability. This further increases the cost of liquefaction.

In U.S. Pat. No. 6,093,562, variants of a parent alpha amylase, in which variant at least one amino acid residue of the parent alpha amylase has been deleted, the variant having alpha amylase activity and increased thermostability. One of the parent alpha amylases being obtainable from Bacillus stearothermophilus and was described in, inter alia, J. Bacteriol. 166 (1986) pp. 635-643.

In J. Biol. Chem. 264(32), at pages 18933-18938 (1989), by Suzuki, et al, the thermostabilities of alpha amylases with amino acid alterations in regions 176-178 (corresponding to residues 179-181 of Bacillus stearothermophilus, SEQ ID. NO.: 3) and 266-269 (corresponding to residues 269-272 of Bacillus stearothermophilus, SEQ ID. NO.:3) were described.

Studies using recombinant DNA techniques to explore which residues are important for the catalytic activity of amylases and/or to explore the effect of modifying certain amino acids within the active site of various amylases and glycosylases have been conducted by various researchers (Vihinen et al., J. Biochem., Vol. 107, pp. 267-272 (1990); Holm et al., Protein Engineering, Vol. 3, pp. 181-191 (1990); Takase et al., Biochemica et Biophysica Acta, Vol. 1120, pp. 281-288 (1992); Matsui et al., FEBS Letters, Vol. 310, pp. 216-218 (1992); Matsui et al., Biochemistry, Vol. 33, pp. 451-458 (1992); Sogaard et al., J. Biol. Chem., Vol. 268, pp. 22480-22484 (1993); Sogaard et al., Carbohydrate Polymers, Vol. 21, pp. 137-146 (1993); Svensson, Plant Mol. Biol., Vol. 25, pp. 141-157 (1994); Svensson et al., J. Biotech., Vol. 29, pp. 1-37 (1993)). Researchers have also studied which residues are important for thermal stability (Suzuki et al., J. Biol. Chem. Vol. 264, pp. 18933-18938 (1989); Watanabe et al., Eur. J. Biochem., Vol. 226, pp. 277-283 (1994)); and one group has used such methods to introduce mutations at various histidine residues in a Bacillus licheniformis amylase, the rationale being that Bacillus licheniformis amylase which is known to be relatively thermostable when compared to other similar Bacillus amylases, has an excess of histidines and, therefore, it was suggested that replacing a histidine could affect the thermostability of the enzyme. This work resulted in the identification of stabilizing mutations at the histidine residue at the +133 position and the alanine residue at position +209 (Declerck et al., J. Biol. Chem., Vol. 265, pp. 15481-15488 (1990); FR 2 665 178-A1; Joyet et al., Bio/Technology, Vol. 10, pp. 1579-1583 (1992)).

Despite the advances made in the prior art, a need exists for an α-amylase which is more effective in commercial liquefaction processes but allowing activity at lower pH than currently practical. Additionally, a need exists for improved amylases having characteristics which makes them more effective under the conditions of detergent use. Because commercially available amylases are not acceptable under many conditions due to stability problems, for example, the high alkalinity and oxidant (bleach) levels associated with detergents, or temperatures under which they operate, there is a need for an amylase having altered, and preferably increased, performance profiles under such conditions.

### SUMMARY OF THE INVENTION

It is an object of the present invention to provide an α-amylase having altered performance profiles.

It is a further object of the present invention to provide an α-amylase having improved stability at high temperature.

Accordingly the present invention provides a variant of a precursor Bacillus stearothermophilus alpha amylase comprising deletions at one or more of the following positions R179 and G180 of the amino acid sequence shown in SEQ ID NO.:3 and/or in a corresponding position in an alpha amylase which displays at least 90% identity with the amino acid sequence of SEQ ID NO.:3. In another embodiment of the present invention, a variant of a precursor Bacillus stearothermophilus alpha amylase comprises deletions at positions R179 and G180 of the amino acid sequence shown in SEQ ID NO.:3 and/or in a corresponding position in an alpha amylase which displays at least 90% identity with the amino acid sequence of SEQ ID NO.2. In another embodiment of the present invention, a DNA is provided that encodes the variant alpha amylase. In another embodiment of the present invention, an expression vector is provided comprising the DNA described above. In another embodiment, a host cell is provided that is transformed with the expression vector of the described above. In another embodiment, the host cell is a Bacillus sp. In another embodiment, the Bacillus species is selected from the group of Bacillus subtilis and Bacillus licheniformis. Another aspect of the present invention provides a detergent composition comprising the variant alpha amylase described above. Another aspect of the present invention provides a starch liquefying composition comprising the variant alpha amylase described above. Another aspect of the present invention provides a method of liquefying starch comprising the steps of contacting a slurry of starch with a variant α-amylase comprising the deletions described above, raising the temperature of the slurry to 60 to 80 C; and maintaining the viscosity of the slurry below 200.0 Ncm. Another aspect of the present invention provides a method of liquefying starch comprising the steps of contacting a slurry of starch with a variant α-amylase comprising the deletions described above, raising the temperature of the slurry to 85 to 100° C.; and providing an average DE progression of at least 8.00 within 60 minutes of the onset of secondary liquefaction.

In an embodiment of this aspect, the α-amylase will be truncated. In some embodiments, the truncated α-amylase comprises a sequence of SEQ ID NO:16 (as shown in FIG. 14) or a sequence having at least 97% sequence identity thereto. In an embodiment expression constructs comprise a DNA sequence encoding the truncated α-amylase. In an embodiment vectors comprise a DNA sequence encoding the truncated α-amylase. In an embodiment compositions comprise the truncated α-amylase. In an embodiment compositions comprising the truncated α-amylase are used in a method of liquefying starch comprising the steps of contacting a slurry of starch with a truncated α-amylase comprising the deletions described above, raising the temperature of the slurry to 60 to 80° C.; and maintaining the viscosity of the slurry below 200.0 Ncm. Another aspect of the present invention provides a method of liquefying starch comprising the steps of contacting a slurry of starch with a truncated α-amylase comprising the deletions described above, raising the temperature of the slurry to 85 to 100° C.; and providing an average DE progression of at least 8.00 within 60 minutes of the onset of secondary liquefaction.

### BRIEF DESCRIPTION OF THE DRAWINGS

FIG. 1 illustrates the DNA sequence of the gene for α-amylase from Bacillus stearothermophilus (SEQ ID NO:1)

FIG. 2 illustrates the pro-form of the alpha amylase amino acid sequence of the B. stearothermophilus (SEQ ID NO:2). The signal sequence is underlined and in bold.

FIG. 3 illustrates the amino acid sequence (SEQ ID NO:3) of the mature α-amylase enzyme from Bacillus stearothermophilus. Amino acid residues R179 and G180 are underlined and in bold.

FIG. 4 illustrates the amino acid sequence (SEQ ID NO 4) of the variant alpha amylase (VAA)

FIG. 5 illustrates an alignment of the primary structures of four Bacillus α-amylases. The variant alpha amylase of the present invention (VAA). The Bacillus licheniformis α-amylase (Am-Lich) (SEQ ID NO:5) is described by Gray et al., J. Bacteriology, Vol. 166, pp. 635-643 (1986); the Bacillus amyloliquefaciens α-amylase (Am-Amylo) (SEQ ID NO:6) is described by Takkinen et al., J. Biol. Chem., Vol. 258, pp. 1007-1013 (1983); the Bacillus stearothermophilus α-amylase (Am-Stearo) (SEQ ID NO:7) is described by Gray et al., J. Bacteriology, Vol. 166, pp. 635-643 (1986).

FIGS. 6A-6B-2 illustrate a fusion protein with the signal peptide of B. licheniformis α-amylase (LAT). The LAT signal peptide is in bold and underlined.

FIG. 7 depicts the plasmid pHPLT

FIG. 8 depicts the plasmid pHPLT-VAAc1.

FIG. 9 illustrates halo formation after 16 hours growth at 37° C. on starch plates (HI-agar/neomycin/0.2% starch) after iodine staining by variant alpha amylases secreted by Neomycin resistant transformants.

FIG. 10 illustrates plasmid plCatH-VAAc1 (Ori2) wherein ori pE 194 (ts) refers to the origin of replication from plasmid plCatH.

FIG. 11 is a graph illustrating the DE progression of the slurry over time of a Wild type or native B. stearothermophilus (2 A-10 u/gm or 0.28 kg/MT dry solids) (-▪-) and variant alpha amylase of the present invention (-♦-)

FIG. 12 is a graph illustrating the slurry viscosity progression of a B. stearothermophilus variant (-♦-), variant alpha amylase of the present inventions (-▴-), and variant B. licheniformis (-x-) (4 A-10 u/gm or 0.56 kg/MT dry solids) as a measure of viscosity (Ncm) over time (min).

FIG. 13 depicts the intact molecular weight measurement of the alpha amylase produced in Example 1 by mass spectrometer. The apparent molecular weight matches to the predicted amino acid sequence of a truncated alpha amylase, e.g., amino acid residues 1-484 of SEQ ID NO:3.

FIG. 14 illustrates the peptide mapping results for the amino acid sequence (SEQ ID NO:16) of the truncated alpha amylase (tAA).

### DETAILED DESCRIPTION

A. Definitions

All patents and publications, including all sequences disclosed within such patents and publications, referred to herein are expressly incorporated by reference. Unless defined otherwise, all technical and scientific terms used herein have the same meaning as commonly understood by one of ordinary skill in the art to which this invention belongs (See e.g., Singleton et al., Dictionary of Microbiology and Molecular Biology, 2d Ed., John Wiley and Sons, New York [1994]; and Hale and Marham, The Harper Collins Dictionary of Biology, Harper Perennial, N.Y. [1991], both of which provide one of skill with a general dictionary of may of the terms used herein). Although any methods and materials similar or equivalent to those described herein can be used in the practice or testing of the present invention, the preferred methods and materials are described. Numeric ranges are inclusive of the numbers defining the range. As used herein and in the appended claims, the singular “a”, “an” and “the” includes the plural reference unless the context clearly dictates otherwise. Thus, for example, reference to a “host cell” includes a plurality of such host cells.

Unless otherwise indicated, nucleic acids are written left to right in 5′ to 3′ orientation; amino acid sequences are written left to right in amino to carboxy orientation, respectively. The headings provided herein are not limitations of the various aspects or embodiments of the invention that can be had by reference to the specification as a whole. Accordingly, the terms defined immediately below are more fully defined by reference to the Specification as a whole.

The term “alpha-amylase (e.g., E.C. class 3.2.1.1)” refers to enzymes that catalyze the hydrolysis of alpha-1,4-glucosidic linkages. These enzymes have also been described as those effecting the exo or endohydrolysis of 1,4-α-D-glucosidic linkages in polysaccharides containing 1,4-α-linked D-glucose units. Another term used to describe these enzymes is “glycogenase”. Exemplary enzymes include alpha-1,4-glucan 4-glucanohydrase glucanohydrolase.

As used herein, “recombinant α-amylase” refers to an α-amylase in which the DNA sequence encoding the naturally occurring α-amylase is modified to produce a mutant DNA sequence which encodes the substitution, insertion or deletion of one or more amino acids in the α-amylase sequence compared to the naturally occurring α-amylase.

The terms “recombinantly expressed α-amylase” and “recombinantly produced α-amylase” refer to a mature α-amylase protein sequence that is produced in a host cell from the expression of a heterologous polynucleotide. For example, the term “r-α-amylase” means the alpha amylase (e.g., SEQ ID NO: 3 or 16) is expressed and produced in a host in which a polynucleotide encoding the α-amylase has been introduced. The mature protein sequence of a r-AA excludes a signal sequence.

The term “recombinant” when used in reference to a cell, nucleic acid, protein or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “protein” and “polypeptide” are used interchangeably herein. The conventional one-letter or three-letter code for amino acid residues is used herein.

A “signal sequence” means a sequence of amino acids bound to the N-terminal portion of a protein, which facilitates the secretion of the mature form of the protein outside the cell. The definition of a signal sequence is a functional one. The mature form of the extracellular protein lacks the signal sequence which is cleaved off during the secretion process.

As used herein, “precursor α-amylase” refers to an alpha amylase in which the DNA sequence is that which encodes the naturally occurring alpha amylase or starting DNA sequence has not yet been modified as described in this application. Thus precursor protease may include known wild type amino acid sequences or modified amino acid sequences other than the modifications described herein, e.g., having altered sequences from wild type in addition to the deletions described herein.

As used herein, the terms “wild type” or “native α-amylase” refer to an alpha amylase in which the DNA sequence is that which encodes the naturally occurring alpha amylase or starting DNA sequence has not yet been modified.

As used herein, the term “pro” form of an amylase refers to a form of the amylase having an additional amino acid/nucleotide sequence operably linked to the amino terminus of the protein and/or a signal sequence operably linked to the amino terminus of the prosequence.

As used herein, the term “variant alpha amylase” (VAA) refers to an alpha amylase in which the DNA sequence that encodes a precursor alpha amylase has been modified to produce a mutant DNA sequence which encodes an amino acid sequence different from the precursor alpha amylase amino acid sequence. For example a variant alpha amylase can comprise an amino acid sequence comprising the deletion of residue positions 179 and/or 180 of SEQ ID. NO.:3.

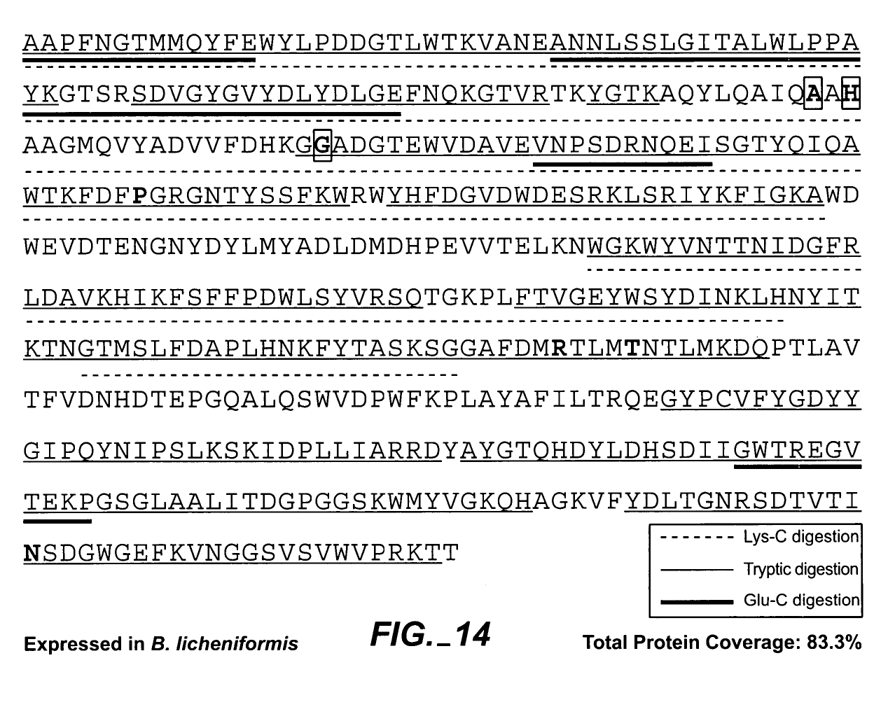

The term “truncated α-amylase” refers to an α-amylase and includes a polypeptide having an amino acid sequence which comprises at least 65% of the amino acid sequence of SEQ ID NO:3 or an amino acid sequence having at least 90% sequence identity to SEQ ID NO:16 (as shown in FIG. 14) wherein part of the SBD has been eliminated, e.g., removed, deleted or the like.

The term “starch binding domain (SBD)” refers to an amino acid sequence that binds preferentially to a starch (polysaccharide) substrate.

The term “linker” refers to a short amino acid sequence generally having between 3 and 40 amino acid residues which covalently binds an amino acid sequence comprising a starch binding domain with an amino acid sequence comprising a catalytic domain.

The term “catalytic domain” refers to a structural region of a polypeptide which is distinct from the SBD and which contains the active site for substrate hydrolysis.

A “deletion” of an amino acid as used herein refers to a modification of the amino acid sequence of the precursor α-amylase which results in the removal of amino acid positions of the precursor amylase, but preferably refers to using genetic engineering to mutate a nucleic acid encoding the precursor α-amylase so as to delete the respective residue in the expressed protein.

A deletion of a consecutive stretch of amino acid residues, exemplified by amino acid residues 30-33, is indicated as (30-33)*.

A deletion of a specific amino acid residue, exemplified by a deletion of the amino acid residue at position 179, is indicated as: Arg179* or R179*.

As used herein, the term “derived from” refers to the source of the precursor alpha amylase which encodes the precursor alpha amylase. Thus an alpha amylase derived from a source includes those isolated from a particular source microorganism. In addition, an alpha amylase derived from a source includes those encoded by or expressed from DNA originating from the source organism.

As used herein the terms “substantially similar” and “substantially identical” in the context of two nucleic acids or polypeptides typically refers to a polynucleotide or polypeptide comprises a sequence that has at least 75% sequence identity, preferably at least 80%, more preferably at least 90%, still more preferably 95%, most preferably 97%, sometimes as much as 98% and 99% sequence identity, compared to the reference (i.e., wild-type) sequence. Sequence identity may be determined using known programs such as BLAST, ALIGN, and CLUSTAL using standard parameters. (See e.g., Altschul, et al., J. Mol. Biol. 215:403-410 [1990]; Henikoff et al., Proc. Natl. Acad. Sci. USA 89:10915 [1989]; Karin et al., Proc. Natl. Acad. Sci USA 90:5873 [1993]; and Higgins et al., Gene 73:237-244 [1988]). Software for performing BLAST analyses is publicly available through the National Center for Biotechnology Information.

As used herein, the terms “percent (%) nucleic acid sequence identity”, “percent (%) nucleotide identity”, “percent (%) amino acid sequence identity” or “percent (%) sequence identity” refer to the percentage of nucleic acid, nucleotide or amino acid residues in a candidate sequence that are identical with the nucleic acid, nucleotide or amino acid residues of the sequence being compared with.

A polynucleotide or a polypeptide having a certain percent (e.g. 80%, 85%, 90%, 95%, or 99%) of sequence identity with another sequence means that, when aligned, that percentage of bases or amino acid residues are the same in comparing the two sequences. This alignment and the percent homology or identity can be determined using any suitable software program known in the art, for example those described in CURRENT PROTOCOLS IN MOLECULAR BIOLOGY (F. M. Ausubel et al. (eds) 1987, Supplement 30, section 7.7.18). Preferred programs include the GCG Pileup program, FASTA (Pearson et al. (1988) Proc. Natl, Acad. Sci USA 85:2444-2448), and BLAST (BLAST Manual, Altschul et al., Natl. Cent. Biotechnol. Inf., Natl Lib. Med. (NCIB NLM NIH), Bethesda, Md., and Altschul et al., (1997) NAR 25:3389-3402). Another preferred alignment program is ALIGN Plus (Scientific and Educational Software, PA), preferably using default parameters. Another sequence software program that finds use is the TFASTA Data Searching Program available in the Sequence Software Package Version 6.0 (Genetics Computer Group, University of Wisconsin, Madison, Wis.).

As used herein, “corresponding to,” refers to a residue at the enumerated position in a first protein or peptide, or a residue that is equivalent to an enumerated residue in a second protein or peptide. Equivalent enumerated residues can be determined by alignment of candidate sequences using the degree of homology programs described above.

A “vector” refers to a polynucleotide sequence designed to introduce nucleic acids into one or more cell types. Vectors include cloning vectors, expression vectors, shuttle vectors, plasmids, phage particles, cassettes and the like.

As used herein, the term “expression vector” refers to any nucleic acid that can be replicated in cells and can carry new genes or DNA segments into cells. Thus the term refers to a nucleic acid construct designed for transfer between different host cells. An expression vector refers to a vector that has the ability to incorporate and express heterologous DNA fragments in a foreign cell. Thus, an “expression vector” as used herein means a DNA construct comprising a DNA sequence which is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

A “promoter” is a regulatory sequence that is involved in binding RNA polymerase to initiate transcription of a gene. The promoter may be an inducible promoter or a constitutive promoter. A preferred promoter used in the invention is Trichoderma reesei cbh1, which is an inducible promoter.

“Under transcriptional control” is a term well understood in the art that indicates that transcription of a polynucleotide sequence, usually a DNA sequence, depends on its being operably linked to an element which contributes to the initiation of, or promotes transcription.

“Under translational control” is a term well understood in the art that indicates a regulatory process that occurs after mRNA has been formed.

The term “derived” encompasses the terms “originated from”, “obtained” or “obtainable from”, and “isolated from”.

The term “operably linked” refers to juxtaposition wherein the elements are in an arrangement allowing them to be functionally related. For example, a promoter is operably linked to a coding sequence if it controls the transcription of the sequence.

The term “selective marker” refers to a gene capable of expression in a host that allows for ease of selection of those hosts containing an introduced nucleic acid or vector. Examples of selectable markers include but are not limited to antimicrobials (e.g., hygromycin, bleomycin, or chloramphenicol) and/or genes that confer a metabolic advantage, such as a nutritional advantage on the host cell.

As used herein, the terms “recovered”, “isolated” and “purified” refer to a nucleic acid or amino acid (or other component) that is removed from at least one component with which it is naturally associated.

As used herein, the terms “host strain” or “host cell” refers to a suitable host for an expression vector or DNA construct comprising DNA encoding the α-amylase according to the present invention.

As used herein, the terms “transformed”, “stably transformed” and “transgenic” used in reference to a cell means the cell has a non-native (e.g., heterologous) nucleic acid sequence integrated into its genome or as an episomal plasmid that is maintained through multiple generations.

As used herein, a “thermostable” amylase refers to an amylase that maintains a greater amount of enzymatic activity as compared to the precursor amylase under the same thermal conditions. For example, a thermostable amylase has an increased level of enzymatic activity of the variant as compared to the precursor at a given temperature, typically the operation temperature of as measured.

The term “contacting” refers to the placing of the respective enzyme(s) in sufficiently close proximity to the respective substrate to enable the enzyme(s) to convert the substrate to the end-product. Those skilled in the art will recognize that mixing solutions of the enzyme with the respective substrates can effect contacting.

The term “heterologous” with reference to a polynucleotide or protein refers to a polynucleotide or protein that does not naturally occur in a host cell. In some embodiments, the protein is a commercially important industrial protein. It is intended that the term encompass proteins that are encoded by naturally occurring genes, mutated genes, and/or synthetic genes.

The term “endogenous” with reference to a polynucleotide or protein refers to a polynucleotide or protein that occurs naturally in the host cell.

As used herein, a “viscosity reducing” amylase refers to an amylase that minimizes the slurry viscosity as it approaches gelatinizing temperatures, e.g., raising the slurry temperatures from 60° to 95° C. For example, a viscosity reducing amylase maintains the viscosity of the slurry to less than a specified amount, e.g., 190.0 Ncm, less than 200.0 Ncm, less than 220 Ncm.

“Ncm” unit refers to a measurement of viscosity of a fluid as a torque measurement using a viscometer.

As used herein, the term amylase activity refers to the rate of starch hydrolysis, as reflected in the rate of decrease in iodine-staining capacity, which is measured spectrophotometrically. One unit of bacterial alpha amylase activity is the amount of enzyme required to hydrolyze 10 mg of starch per minute under specified conditions. For example, 0.14 kg/MT dry VAA.

As used herein, the term “average DE progression” refers to the amount of DE produced over a given amount of time.

As used herein, the term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100. An exemplarly method for determining the DE of a slurry or solution is described in Schroorl's method (Fehling's assay titration) (see Example 3a).

As used herein, the term “average DE progression” refers to the change in DE as a function of time of secondary liquefaction. The slope of a DE versus minutes of liquefaction is a measure of the speed a DE level is achieved.

As used herein, the term “liquefaction” or “liquefy” means a process by which starch is converted to shorter chain and less viscous dextrins. Generally, this process involves gelatinization of starch simultaneously with or followed by the addition of α-amylase.

As used herein, the term “primary liquefaction” refers to a step of liquefaction when the slurry's temperature is raised to or near its gelatinization temperature. Subsequent to the raising of the temperature, the slurry is sent through a heat exchanger or jet to temperatures from 200-300° F., e.g., 220-235 degrees F. Subsequent to application to a heat exchange or jet temperature, the slurry is held for a period of 3-10 minutes at that temperature. This step of holding the slurry at 200-300° F. is primary liquefaction.

As used herein, the term “secondary liquefaction” refers the liquefaction step subsequent to primary liquefaction (heating to 200-300° F.) when the slurry is allowed to cool to atmospheric temperature. This cooling step can be 30 minutes to 180 minutes (3 hours), e.g. 90 minutes to 120 minutes (2 hours).

As used herein, the term “minutes of secondary liquefaction” refers to the time that has elapsed from the start of secondary liquefaction, time that the DE is measured.

### Embodiments

Suitable Amylase Sources

The precursor α-amylase is produced by any source capable of producing α-amylase. Suitable sources of α-amylases are prokaryotic or eukaryotic organisms, including fungi, bacteria, plants or animals. Preferably, the precursor α-amylase is derived from a Bacillus; more preferably, by Bacillus licheniformis, Bacillus amyloliquefaciens or Bacillus stearothermophilus; more preferably, the precursor α-amylase is derived from Bacillus stearothermophilus or Geobacillus stearothermophilus (SEQ ID NO.:3). Modification of the precursor DNA sequence which encodes the amino acid sequence of the precursor α-amylase can be by methods described herein and in commonly owned U.S. Pat. Nos. 4,760,025 and 5,185,258, incorporated herein by reference.

In another embodiment, the precursor α-amylase equivalent to the mature Geobacillus stearothermophilus in FIG. 3, has a % amino acid sequence identity of at least 75%, 80%, 85%,90%, 92% 95%, 96%, 97%, 98%, 99% of SEQ ID NO. 3. In another embodiment, the precursor α-amylase has an identical amino acid sequence to the amino acid sequence of the mature Bacillus stearothermophilus in FIG. 3 (SEQ ID NO.:3).

In another embodiment, the precursor alpha amylase comprises an amino acid sequence further comprising at least 4 consecutive amino acids that are identical to SEQ ID NO. 10, the amino acid sequence corresponding to amino acids 269-272 of SEQ ID NO 3. In one embodiment, the 4 consecutive amino acids comprise the sequence DINK, e.g, Asp269 (D269), Iso270 (I270), Asn271 (N271) and Lys272 (K272).

In another embodiment, the precursor amylase comprises an amino acid sequence further comprising at least 4 consecutive amino acids that are identical to SEQ ID NO 11, the amino acid sequence corresponding to amino acids 178 and 181-183 of SEQ ID NO. 3. In one embodiment, the 4 consecutive amino acids comprise the sequence FIGK, e.g., Phe178 (F178)-Iso181 (1181)-Gly182 (G182)—Lys183 (K183).

In another embodiment, the precursor amylase comprises an amino acid sequence further comprising at least 4 consecutive amino acids that are identical to SEQ ID NO 12, the amino acid sequence corresponding to amino acids 301 to 304 of SEQ ID NO. 3. In one embodiment, the four consecutive amino acids comprise the sequence Gly301 (G301)-ala302 (A302)-phe303 (F303)—asp304 (D304).

In another embodiment, the precursor amylase comprises an amino acid sequence further comprising at least 5 consecutive amino acids that are identical to SEQ ID NO 13, the amino acid sequence corresponding to amino acids 412 to 416 of SEQ ID NO. 3. In one embodiment, the five consecutive amino acids comprise the amino acid sequence EGGTE, e.g., glu412 (E412)-Gly413 (G413)-Gly414 (G414)—Thr415 (T415)-glu416 (E416).

In another embodiment, the precursor amylase comprises an amino acid sequence further comprising at least 4 consecutive amino acids that are identical to SEQ ID NO 14, the amino acid sequence corresponding to amino acids 489 to 492 of SEQ ID NO. 3. In one embodiment, the four consecutive amino acids comprise the sequence ARPI, e.g., Ala489 (“A489”)-arg490 (“R490”)-pro491 (“P491”)—iso492 (“1492”).

In another embodiment, the precursor amylase comprises an amino acid sequence further comprising at least 4 consecutive amino acids that are identical to SEQ ID NO 15, the SEQ ID comprising amino acids 498 to 501 of SEQ ID NO. 3. In one embodiment, the four consecutive amino acids comprise the sequence TGEF, e.g., Thr498 (T498)-Gly499 (G499)-Glu500 (E500)—phe501 (F501).

In another embodiment, the precursor amylase comprises an amino acid sequence containing at least one, at least two, at least 3, at least 4 and at least 5 acid sequences that are identical to the corresponding amino acid sequences:
(a) FIGK, (b) GAFD; (c) EGGTE; (d) ARPI; (e) TGEF and (f) DINK.

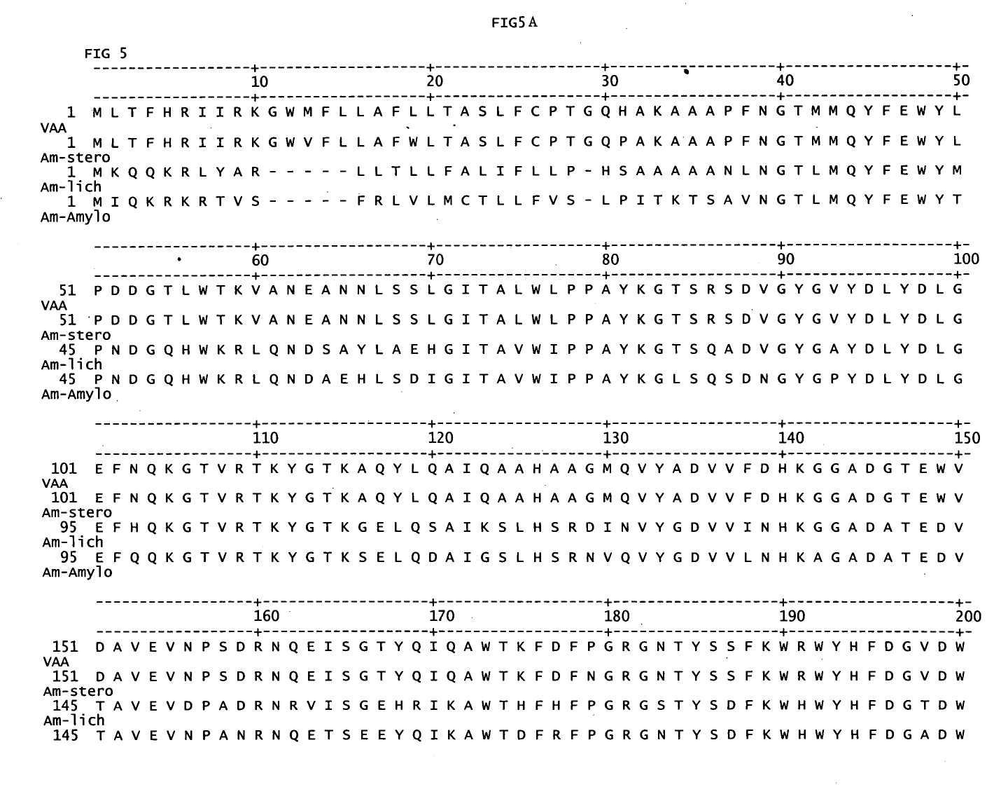

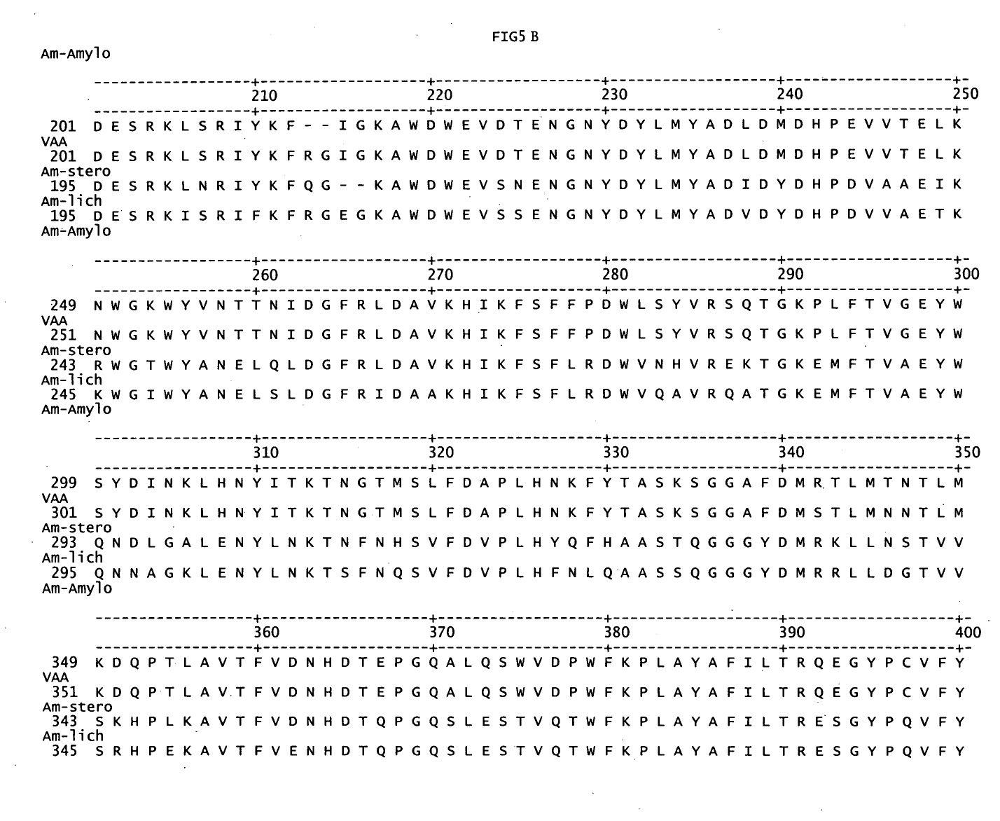

Homologies have been found between almost all endo-amylases sequenced to date, ranging from plants, mammals, and bacteria (Nakajima et al., Appl. Microbiol. Biotechnol., Vol. 23, pp. 355-360 (1986); Rogers, Biochem. Biophys. Res. Commun., Vol. 128, pp. 470-476 (1985); Janecek, Eur. J. Biochem., Vol. 224, pp. 519-524 (1994)). There are four areas of particularly high homology in certain Bacillus amylases, as shown in FIG. 5. Sequence alignments have also been used to map the relationship between Bacillus endo-amylases (Feng et al., J. Molec. Evol., Vol. 35, pp. 351-360 (1987)). The relative sequence homology between Bacillus stearothermophilus and Bacillus licheniformis amylase is about 66% and that between Bacillus licheniformis and Bacillus amyloliquefaciens amylases is about 81%, as determined by Holm et al., Protein Engineering, Vol. 3, No. 3, pp. 181-191 (1990). While sequence homology is important, it is generally recognized that structural homology is also important in comparing amylases or other enzymes. For example, structural homology between fungal amylases and bacterial amylase has been suggested and, therefore, fungal amylases are encompassed within the present invention.

In order to establish homology to primary structure, the amino acid sequence of a precursor α-amylase is directly compared to the Bacillus stearothermophilus α-amylase primary sequence and particularly to a set of residues known to be invariant to all α-amylases for which sequences are known (see e.g., FIG. 3). It is possible also to determine equivalent residues by tertiary structure analysis of the crystal structures reported for porcine pancreatic α-amylase (Buisson et al., EMBO Journal, Vol. 6, pp. 3909-3916 (1987); Qian et al., Biochemistry, Vol. 33, pp. 6284-6294 (1994); Larson et al., J. Mol. Biol., Vol. 235, pp. 1560-1584 (1994)); Taka-amylase A from Aspergillus oryzae (Matsuura et al., J. Biochem. (Tokyo), Vol. 95, pp. 697-702 (1984)); and an acid α-amylase from A. niger (Boel et al. Biochemistry, Vol. 29, pp. 6244-6249 (1990)), with the former two structures being similar, and for barley α-amylase (Vallee et al., J. Mol. Biol., Vol. 236, pp. 368-371 (1994); Kadziola, J. Mol. Biol., Vol. 239, pp. 104-121 (1994)). Several preliminary studies have been published related to the secondary structure of α-amylase, i.e., (Suzuki et al., J. Biochem., Vol. 108, pp. 379-381 (1990); Lee et al., Arch. Biochem. Biophys, Vol. 291, pp. 255-257 (1991); Chang et al., J. Mol. Biol., Vol. 229, pp. 235-238 (1993); Mizuno et al., J. Mol. Biol., Vol. 234, pp. 1282-1283 (1993)), and at least one structure has been published for crystalline Bacillus stearothermophilus α-amylase (Machius et al., J. Mol. Biol. Vol. 246, pp. 545-549 (1995)). However, several researchers have predicted common super-secondary structures between glucanases (MacGregor et al., Biochem. J., Vol. 259, pp. 145-152 (1989)) and within α-amylases and other starch-metabolising enzymes (Jaspersen, J. Prot. Chem. Vol. 12, pp. 791-805 (1993); MacGregor, Starke, Vol. 45, pp. 232-237 (1993)); and sequence similarities between enzymes with similar super-secondary structures to α-amylases (Janecek, FEBS Letters, Vol. 316, pp. 23-26 (1993); Janecek et al., J. Prot. Chem., Vol. 12, pp. 509-514 (1993)). A structure for the Bacillus stearothermophilus enzyme has been modeled on that of Taka-amylase A (Holm et al., Protein Engineering, Vol. 3, pp. 181-191 (1990)). The four highly conserved regions shown in FIG. 3 contain many residues thought to be part of the active-site (Matsuura et al., J. Biochem. (Tokyo), Vol. 95, pp. 697-702 (1984); Buisson et al., EMBO Journal, Vol. 6, pp. 3909-3916 (1987); Vihinen et al., J. Biochem., Vol. 107, pp. 267-272 (1990)) including His +105; Arg +229; Asp +231; His +235; Glu +261 and Asp +328 under the Bacillus licheniformis numbering system.

The degree of homology between sequences may be determined using any suitable method known in the art (See e.g., Smith and Waterman, Adv. Appl. Math., 2:482 [1981]; Needleman and Wunsch, J. Mol. Biol., 48:443 [1970]; Pearson and Lipman, Proc. Natl. Acad. Sci. USA 85:2444 [1988]; programs such as GAP, BESTFIT, FASTA, and TFASTA in the Wisconsin Genetics Software Package (Genetics Computer Group, Madison, Wis.); and Devereux et al., Nucl. Acid Res., 12:387-395 [1984]).

For example, PILEUP is a useful program to determine sequence homology levels. PILEUP creates a multiple sequence alignment from a group of related sequences using progressive, pairwise alignments. It can also plot a tree showing the clustering relationships used to create the alignment. PILEUP uses a simplification of the progressive alignment method of Feng and Doolittle, (Feng and Doolittle, J. Mol. Evol., 35:351-360 [1987]). The method is similar to that described by Higgins and Sharp (Higgins and Sharp, CABIOS 5:151-153 [1989]). Useful PILEUP parameters including a default gap weight of 3.00, a default gap length weight of 0.10, and weighted end gaps. Another example of a useful algorithm is the BLAST algorithm, described by Altschul et al., (Altschul et al., J. Mol. Biol., 215:403-410, [1990]; and Karlin et al., Proc. Natl. Acad. Sci. USA 90:5873-5787 [1993]). One particularly useful BLAST program is the WU-BLAST-2 program (See, Altschul et al., Meth. Enzymol., 266:460-480 [1996]). parameters “W,” “T,” and “X” determine the sensitivity and speed of the alignment. The BLAST program uses as defaults a word length (W) of 11, the BLOSUM62 scoring matrix (See, Henikoff and Henikoff, Proc. Natl. Acad. Sci. USA 89:10915 [1989]) alignments (B) of 50, expectation (E) of 10, M′5, N′-4, and a comparison of both strands.

An embodiment of the present invention further comprises, in addition to the deletion of residues as provided herein, any one or more of the substitutions known in the art to confer stability or increased activity. In particularly preferred embodiments, the α-amylase according to the present invention may further comprise a deletion or substitution at one or more residues corresponding to M15, A33, A52, S85, N96, V129, H133, S148, S187, N188, A209, A269 and/or A379 in Bacillus licheniformis (SEQ ID NO.:5).

Variant Alpha Amylase

According to the present invention, a variant α-amylase is provided that has introduced therein a deletion corresponding to positions R179* and/or G180* of a precursor Bacillus or Geobacillus alpha amylase, e.g., an amylase having the amino acid sequence of SEQ ID NO:4.

Among others, deletions at residues corresponding to R179 and/or G180 in Bacillus stearothermophilus α-amylase are identified herein. Thus, specific residues such as R179 refer to an amino acid position number (i.e., +179) which references the number assigned to the precursor Bacillus stearothermophilus α-amylase sequence illustrated in FIG. 3 (SEQ ID NO:3). In another embodiment, the invention, however, is not limited to the mutation of the particular precursor α-amylase of Bacillus stearothermophilus but extends to precursor α-amylases containing amino acid residues at positions which correspond to the particular identified residue in Bacillus stearothermophilus α-amylase. Thus, in one embodiment, the R179 of the Bacillus stearotherophilus (Am-ster) of FIG. 5 (SEQ ID NO:7) extends to the a residue of a precursor α-amylase that aligns with R179 of Bacillus stearothermophilus α-amylase. An illustrative alignment is shown in FIG. 5.

In an embodiment a truncated Bacillus stearothermophilus α-amylase having the amino acid sequence shown in FIG. 14 is provided. In one aspect, the α-amylase has the amino acid sequence of SEQ ID NO:16. In another aspect, the α-amylase has at least 97% identity with the amino acid sequence of SEQ ID NO:16.

DNA Encoding

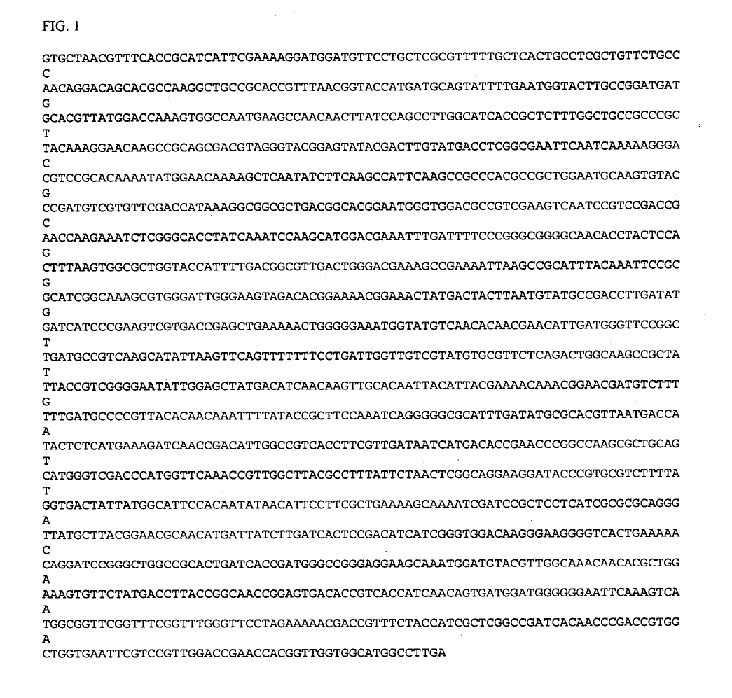

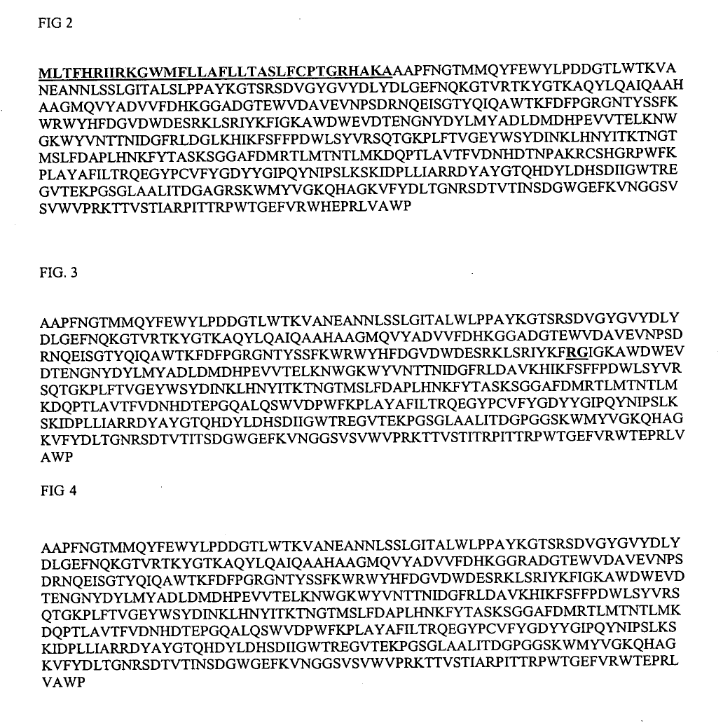

Also provided is a nucleic acid molecule (DNA) which encodes an amino acid sequence comprising the variant α-amylase of the present invention. An additional embodiment of the present invention comprises DNA encoding an α-amylase according to the present invention and expression vectors comprising such DNA. In some embodiments, the transforming DNA comprises an incoming sequence. The ends can be closed such that the transforming DNA forms a closed circle, such as for example, insertion into a vector. In one embodiment, the DNA sequence has a % nucleic acid identity with the nucleic acid sequence of SEQ ID NO.:1 (FIG. 1). In other embodiments, the DNA sequence has at least 75%, 80%, 85%, 88%, 90%, 92%, 95%, 98% nucleic acid identity with the sequence of SEQ ID NO.:1 (FIG. 1). The DNA construct may comprise a DNA sequence which is operably linked to a suitable control sequence capable of effecting the expression of said DNA in a suitable host. The nucleic acid can be generated recombinantly or synthetically, e.g., generated in vitro by PCR. Such control sequences may include a promoter to effect transcription, an optional operator sequence to control such transcription, a sequence encoding suitable mRNA ribosome-binding sites, and sequences which control termination of transcription and translation. The DNA sequences may be expressed by operably linking them to an expression control sequence in an appropriate expression vector and employing that expression vector to transform an appropriate host according to well known techniques. For example, Applicants have discovered that a preferred expression control sequence for Bacillus transformants is the aprE signal peptide derived from Bacillus subtilis when the host cell is B. subtilis. Applicants have also discovered that a preferred expression control sequence for Bacillus licheniformis transformants is the LAT signal peptide derived from Bacillus licheniformis when the host cell is B. licheniformis.

Expression Vectors/Host Cells

Similarly, the present invention includes a method for producing a mutant α-amylase by expressing the DNA incorporated in an expression system which has been transformed into a host cell. A wide variety of host/expression vector combinations may be employed in expressing the DNA sequences of this invention. Many prokaryotic and eukaryotic expression vectors are commercially available. Selection of appropriate expression vectors is within the knowledge of those having skill in the art. The vector may be a plasmid, a phage particle, or simply a potential genomic insert. Once transformed into a suitable host, the vector may replicate and function independently of the host genome, or may, in some instances, integrate into the genome itself. In the present specification, plasmid and vector are sometimes used interchangeably as the plasmid is the most commonly used form of vector at present. However, the invention is intended to include such other forms of expression vectors which serve equivalent functions and which are, or become, known in the art. Useful expression vectors, for example, include segments of chromosomal, non-chromosomal and synthetic DNA sequences, such as the various known plasmids and phages useful for this purpose. In addition, any of a wide variety of expression control sequences are generally used in these vectors.

Host cells useful in the present invention are generally procaryotic or eucaryotic hosts, including any transformable microorganism in which the expression of α-amylase according to the present invention can be achieved. Host cells are transformed or transfected with vectors constructed using recombinant DNA techniques. Such transformed host cells are capable of either replicating vectors encoding the α-amylase and its variants (mutants) or expressing the desired α-amylase. These hosts may include well known eukaryotic and prokaryotic hosts, such as strains of E. coli, Pseudomonas, Bacillus, Streptomyces, various fungi, yeast and animal cells. Preferably, the host expresses the α-amylase of the present invention extracellularly to facilitate purification and downstream processing.

In some preferred embodiments, the host cell is a member of the genus Bacillus, while in some embodiments, the Bacillus strain of interest in an industrial Bacillus strain. Examples of industrial Bacillus strains include, but are not limited to B. licheniformis, B. subtilisis, B lentus, B amyloliquefaciens. In additional embodiments, the Bacillus host strain is selected from the group consisting of B. lentus, B. brevis, B. stearothermophilus, B. alkalophilus, B. coagulans, B. cirulans, B. pumilus, B. thuringiensis, B. clausii, and B. megaterium, as well as as other organisms within the genus Bacillus, as discussed above. In some preferred embodiments, B. subtilis is used. In some particularly preferred embodiments, B. licheniformis is used. For example, U.S. Pat. Nos. 5,264,366 and 4,760,025 (RE34,606), and US2002/0182734 (International Publication No. WO 02/14490) describe various Bacillus host strains that find use in the present invention, although other suitable strains are contemplated for use in the present invention. Preferably, an α-amylase negative Bacillus strain (genes deleted) and/or an α-amylase and protease deleted Bacillus strain (ΔamyE, Δapr, Δnpr) is used.

Transformation

Various methods are known for the transformation of Bacillus species. Indeed, methods for altering the chromosome of Bacillus involving plasmid constructs and transformation of the plasmids into E. coli are well known. In most methods, plasmids are subsequently isolated from E. coli and transformed into Bacillus. However, it is not essential to use such intervening microorganism such as E. coli and in some preferred embodiments, the DNA construct is directly transformed into a competent Bacillus host via protoplasts or competent cell transformation. Expression and purification of the mutant α-amylase of the invention may be effected through art-recognized means for carrying out such processes.

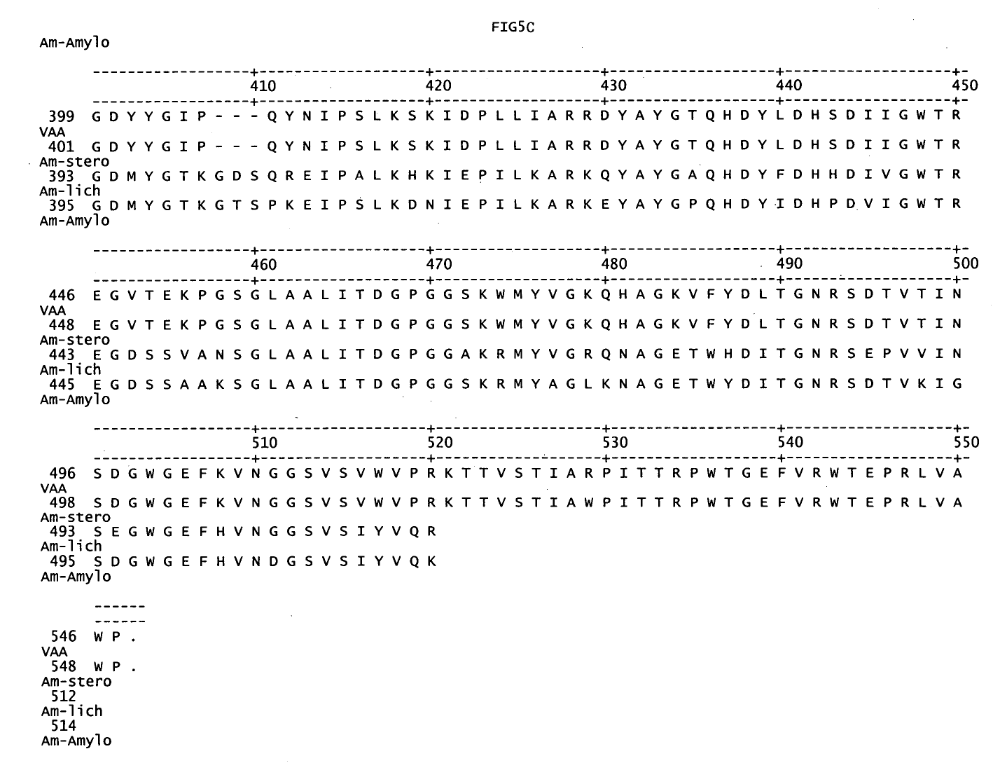

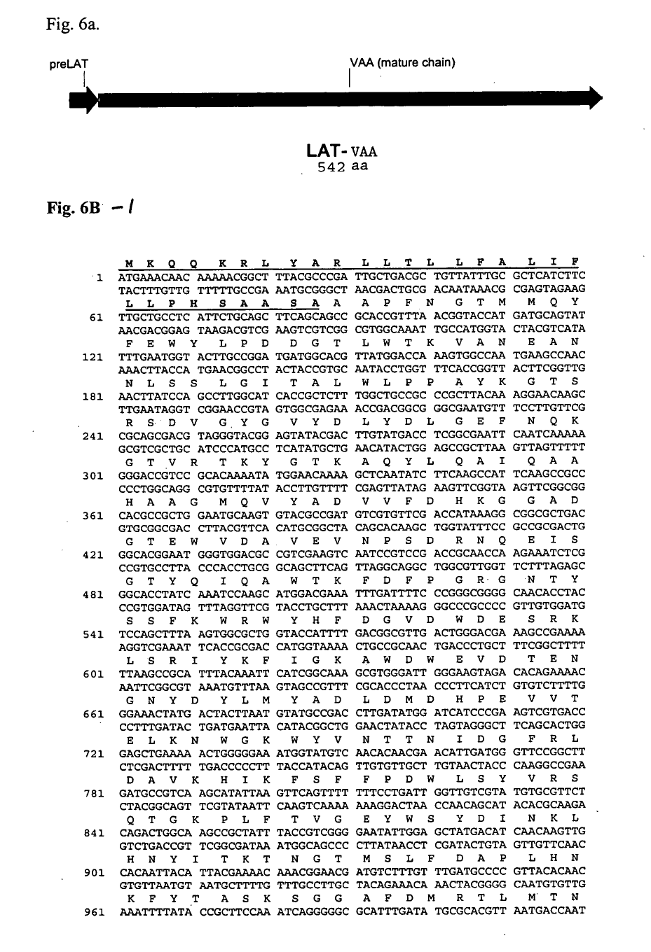

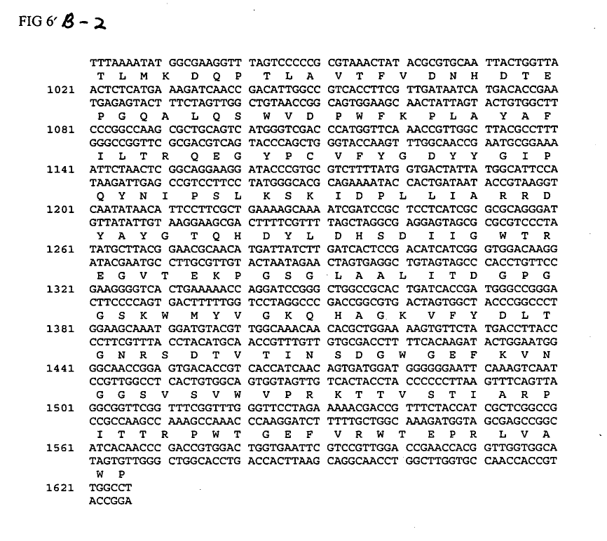

In one embodiment of the present invention, the variant alpha amylase (VAA) is a variant of Geobacillus stearothermophilus α-amylase. In this embodiment, the alpha amylase is expressed in Bacillus licheniformis as a fusion protein with the signal peptide of B. licheniformis α-amylase (LAT) (see FIGS. 6a and 6b). The gene fusion can be created by PCR amplification of the sequence encoding the variant alpha amylase from plasmid pCPCori (obtained from Enzyme BioSystems, Beloit, Wis., USA) and cloning into the vector pHPLT. PCR reactions can be performed on a thermocycler for 30 cycles with a Taq polymerase. pHPLT contains the LAT promoter (P_{LAT}), a sequence encoding the LAT signal peptide (preLAT), followed by PstI and HpaI restriction sites for cloning. The variant alpha amylase was created as PstI-HpaI fragment by fusion PCR (necessary to remove the internal PstI site in the variant alpha amylase gene) with Taq polymerase and the following primers:

VAA(PstI)_FW:

(SEQ ID NO:_{——})

gaatgtctacagcttcagcagccgcaccgtttaacggcaccatg
VAA(Hpal)_RV

(SEQ ID NO:_{——})

cccggggttaactcaaggccatgccaccaaccgtgg
VAAdelPstl_fw

(SEQ ID NO:_{——})

cccggccaagcgcttcagtcatgggtcgac
VAAdelPstl_rv

(SEQ ID NO:_{——})

gtcgacccatgactgaagcgcttggccggg

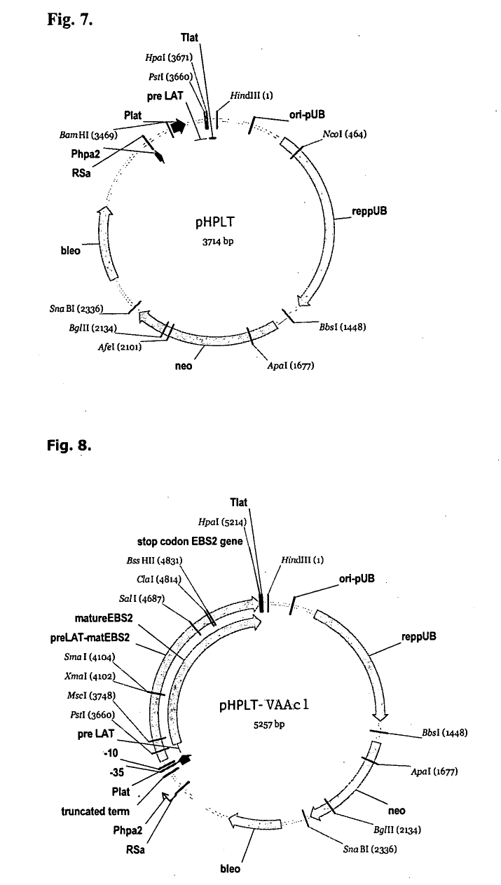

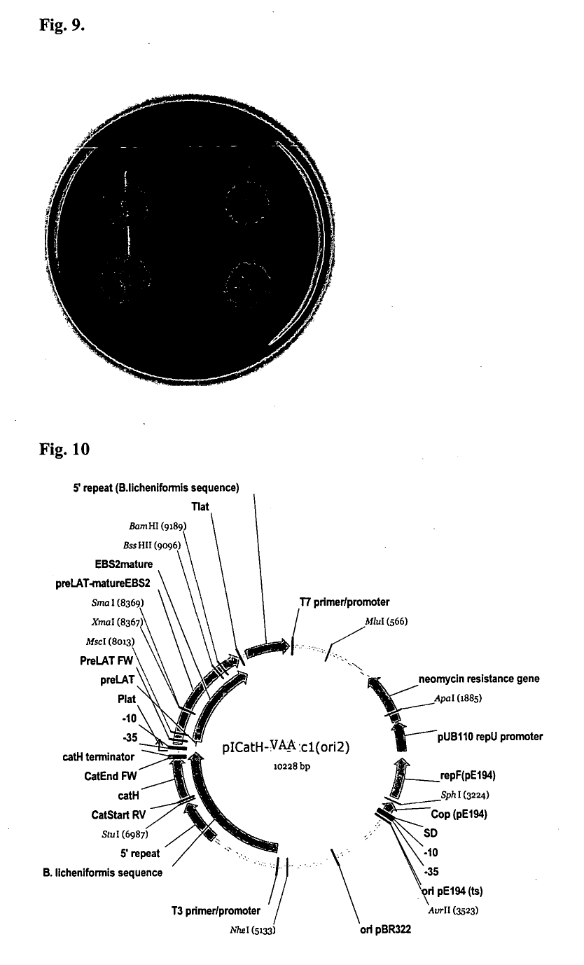

The PstI-HpaI fragment encoding mature alpha amylase is then ligated with T4 DNA ligase into PstI and HpaI digested pHPLT (FIG. 7) and transformed into B. subtilis strain OS14. The sequence of the LAT-VAA gene fusion can be confirmed by DNA sequencing. One of the correct plasmid clones was designated pHPLT-VAAc1 (FIG. 8). This plasmid was introduced into an amylase negative B. licheniformis host (BML612) by protoplast transformation (Pragai et al., Microbiology (1994) 140:305-310). Neomycin resistant transformants secrete variant alpha amylase as judged by halo formation on starch plates after iodine staining (FIG. 9).

Next plasmid plCatH-VAAc1(ori2) (FIG. 10) was created by inserting the LAT-VAA gene construct from pHPLT-VAAc1 into the vector plCatH. plCatH contains the following features: a origin of replication (ori pE194, for replication in Bacillus), ori pBR322 (for amplification in E. coli), a neomycin resistance gene for selection, and the native B. licheniformis chloramphenicol resistance gene (cat) for selection, chromosomal integration and cassette amplification. The preLAT-precursor alpha amylase gene fusion (including the LAT promoter and the LAT transcription terminator) was amplified from pHPLT-VAAc1 with the primers:

(SEQ ID NO:_{——})

| | |
|---|---|
| VAAXhol_FW | atcctactcgaggcttttcttttggaagaaaatataggg |

(SEQ ID NO:_{——})

| | |
|---|---|
| VAAXhol_RV | tggaatctcgaggttttatcctttaccttgtctcc |

The resulting PCR fragment was digested with XhoI, ligated into XhoI digested plCatH and transformed into B. subtilis strain OS14 as described in U.S. Patent Application No. US2002/0182734 (International Publication No. WO 02/14490). Plasmid DNA was isolated from an amylase positive transformant and the sequence of the variant alpha amylase gene construct was confirmed by DNA sequencing. The plasmid of one correct clone was designated plCatH-VAAc1 (ori2) and then transformed into B. licheniformis strain BML612 (BRA7 derivative, cat-, amyL-, spo-) at the permissive temperature (37° C.). One amylase positive, neomycin resistant (neoR) and chloramphenicol resistant (CmR) transformant was selected and designated BML612(plCatH-VAAc1). The plasmid in BML612(pICatH-VMc1) was integrated into the cat region on the B. licheniformis genome by growing the strain at a non-permissive temperature (50° C.) in medium with 5 μg/ml chloramphenicol. One CmR resistant clone was selected and designated BML612-plCatH-VAAc1. BML612-plCatH-VAAc1 was grown again at the permissive temperature for several generations without antibiotics to loop-out vector sequences and then one neomycin sensitive (neoS), CmR clone was selected. In this clone, vector sequences of plCatH on the chromosome are excised (including the neomycin resistance gene) and only the variant alpha amylase-cat cassette is left. Next, the variant alpha amylase-cat cassette on the chromosome was amplified by growing the strain in/on media with increasing concentrations of chloramphenicol. After various rounds of amplification, one clone (resistant against 75 μg/ml chloramphenicol) was selected and designated BML612-VAAc1.

Applications

The variant α-amylase of the invention may be used in liquefaction of starch, as an ingredient in laundry detergents, automatic dishwashing detergents, hard surface cleaning products, in food processing including baking applications, in textile processing including as a desize agent, or in any other application in which α-amylase activity is useful.

α-Amylases according to the present invention which exhibit altered performance characteristics providing desirable and unexpected results are useful in the various applications for which α-amylases are commonly used. For example, α-amylases according to the present invention which exhibit altered performance characteristics e.g., improved viscosity reduction and improved thermostability are useful in operating temperatures used in the liquefaction of starch. Enhanced thermostability will also be useful in extending the shelf life of products which incorporate them. To the contrary, reduced thermal stability may be useful in industrial processes which require the rapid and efficient quenching of amylolytic activity.

α-Amylases of the present invention which exhibit improved thermostability will be especially useful in starch processing and particularly in starch liquefaction. Conditions present during commercially desirable liquefaction processes characteristically include high temperatures requiring α-amylases exhibiting improved thermal stability. Accordingly, α-amylases according to the present invention which are particularly useful in liquefaction exhibit increased thermal stability at temperatures of between about 80-120° C., and preferably between about 100-110° C.

α-Amylases of the present invention which exhibit improved viscosity reduction will also be useful in starch processing and particularly in starch liquefaction. Conditions present during commercially desirable liquefaction processes characteristically include increased viscosity during the gelatinization requiring increased amounts of α-amylases to reduce the slurry viscosity. Accordingly, α-amylases according to the present invention which are particularly useful in liquefaction exhibit increased ability to reduce viscosity, maintains the slurry viscosity below desired levels.

During liquefaction, starch, specifically granular starch slurries from either a wet or dry milled process, is treated with an α-amylase of the present invention according to known liquefaction techniques. Once the slurry has been prepared, in the first step of the starch degradation process, heat is applied to the slurry to gelatinize. Generally, the starch slurry is gelatinized by heating at a relatively high temperature (between about 80° C. and about 110° C.). As the heat rises, the slurry gelatinizes, increasing the viscosity of the slurry. As the viscosity increases, the ability of the slurry to flow decreases. After the starch slurry is gelatinized, it is liquefied using an α-amylase. However, during the liquefaction of the starch, that is when the temperature of the slurry is raised to 60-110, 60-90, 60-85 degrees C., the viscosity of the slurry rises, often times to Ncm levels of 180 or greater Ncm, 190 or greater Ncm, 200 or greater, Ncm levels of 220 or greater Ncm, 240 or greater Ncm, 260 or greater Ncm, 280 or greater Ncm. Contacting the alpha amylase to the increasing viscous starch slurry can reduce the viscosity, a desired benefit. By ascertaining the slurry viscosity progression, one can measure the ability of the particular enzyme to reduce the viscosity of the slurry.

Viscosity can be measured by various means know to those of skill in the art. In one preferred method, the viscosity is measured in a viscometer which comprises a water jacketed sample vessel, and a rotational member for insertion into the sample vessel (Viscoklick. IKA Eurostar Labortechnik power control-visc p7 with a Viscokliick VK1 controller (Werke GMBH & Co, Germany) analyzed on a personal computer with Labworldsoft version 2.6 (Fisher Scientific, GmbH, Germany). A one liter sample of the slurry is placed into the sample vessel. The rotation of the member is set at any speed, so long as the same speed is used to calibrate the torque measured in a control sample. In one embodiment, the rotation is set at 100 rpm. The rotation of the member in the slurry sample, when compared to a control sample is computed by a program (Labworldsoft version 2.6). The temperature of the sample to be tested is raised from 60 to 110, 60 to 90, 60-85 degrees C. in step-wise increments. The amount of torque is then correlated with the control to provide a measurement (Ncm units), which is then recorded at selected time intervals. These numbers are captured and then graphed at appropriate time intervals. Additionally, alpha amylases that provide a quick viscosity break in higher percentage dry solid systems are particularly useful. Additionally, alpha amylases that are useful in pH 5.5-5.8, and/or 5.0 to 6.5

Thus another aspect of the present invention provides a method for liquefying starch comprising the steps of contacting a slurry of starch with a variant α-amylase comprising the deletions described above, raising the temperature of the slurry to 85-100° C., 92-97° C. and/or about 95° C.; and providing an average DE progression of at least a minimum level within 60 minutes of the start of secondary liquefaction.

α-Amylases of the present invention which exhibit improved average DE progression will also be useful in starch processing and particularly in starch liquefaction. Thus another aspect of the present invention provides a method for liquefying starch comprising the steps of contacting a slurry of starch with a variant α-amylase comprising the deletions described above, raising the temperature of the slurry to the levels described above; and providing an increased DE progression within a specified time after the onset of secondary liquefaction.

α-Amylases of the present invention which exhibit improved DE progression will also be useful in starch processing and particularly in starch liquefaction. An increased DE level or a more rapid attainment of an increased DE level could result in better hydrolysis of the substrate. In one embodiment, DE levels can be determined by various methods, e.g., spectrophotometric, gas chromatographic, known to those of skill in the art. One exemplary method is the Schrool method (see Example 3a), wherein the DE is determined at specified predetermined time periods. In one embodiment, the DE is determined at 30 minute intervals. In one embodiment the predetermined sample times begin 30 minutes after the onset of secondary liquefaction. Accordingly, α-amylases according to the present invention which are particularly useful in liquefaction exhibit an increased average DE progression. In various embodiments the alpha amylases of the present invention achieve a DE of at least 7.00, at least 8.00, at least 8.50, at least 9.00 within 60 minutes of the onset of secondary liquefaction.

Additional components known by those skilled in the art to be useful in liquefaction, including, for example, antioxidants, calcium, ions, salts or other enzymes such as endoglycosidases, cellulases, proteases, lipases or other amylase enzymes may be added depending on the intended reaction conditions. For example, combinations of the α-amylase according to the present invention with α-amylases from other sources may provide unique action profiles which find particular use under specific liquefaction conditions. In particular, it is contemplated that the combination of the α-amylase according to the present invention with α-amylase derived from Bacillus stearothermophilus will provide enhanced liquefaction at pH values below 5.5 due to complementary action patterns.

In another aspect of the present invention, detergent compositions in either liquid, gel or granular form, which comprise the variant α-amylase according to the present invention may be useful. Such detergent compositions will particularly benefit from the addition of the variant α-amylase according to the present invention which has increased thermal stability to improve shelf-life. Thus, the variant α-amylase according to the present invention may be advantageously formulated into known powdered, liquid or gel detergents for use in applications having temperatures between about 80° C. and about 100° C. Detergent compositions comprising the variant α-amylase according to the present invention may further include other enzymes such as endoglycosidases, cellulases, proteases, lipases or other amylase enzymes, particularly α-amylase derived from Bacillus licheniformis, amyloliquefaciens, as well as additional ingredients as generally known in the art.

Embodiments of the present invention which comprise a combination of the α-amylase according to the present invention with protease enzymes preferably include oxidatively stable proteases such as those described in U.S. Re. 34,606, incorporated herein by reference, as well as commercially available enzymes such as DURAZYM (Novo Nordisk) and PURAFECT® OxP (Genencor International, Inc.). Methods for making such protease mutants (oxidatively stable proteases), and particularly such mutants having a substitution for the methionine at a position equivalent to M222 in Bacillus amyloliquefaciens, are described in U.S. Re. 34,606.

The variant α-amylases according to the present invention are contemplated to provide important advantages when compared to wild type Bacillus α-amylases. For example, one advantage is the increased activity found at low pH and high temperatures typical of common starch liquefaction methods. Other advantages may include increased high pH and oxidative stability which facilitates their use in detergents; more complete hydrolysis of starch molecules is achieved which reduces residual starch in the processing stream; improved stability in the absence of calcium ion; and that the addition of equal protein doses of α-amylase according to the invention may provide superior performance when compared to wild type Geobacillus stearothermophilus α-amylase due to improvements in both specific activity and stability under stressed conditions.

The following is presented by way of example and is not to be construed as a limitation to the scope of the claims. Abbreviations used herein, particularly three letter or one letter notations for amino acids are described in Dale, J. W., Molecular Genetics of Bacteria, John Wiley & Sons, (1989) Appendix B.

### EXAMPLES

### Example 1

### Expression of the Variant Alpha in Bacillus licheniformis

The variant alpha amylase, a variant of Geobacillus stearothermophilus α-amylase, was expressed in Bacillus licheniformis as a fusion protein with the signal peptide of B. licheniformis α-amylase (LAT) (see FIGS. 6a and 6b). The gene fusion was created by PCR amplification of the sequence encoding the mature chain of the variant alpha amylase from plasmid pCPCori (obtained from Enzyme BioSystems, Beloit, Wis., USA); and cloning into the vector pHPLT. PCR reactions were typically performed on a thermocycler for 30 cycles with High Fidelity Platinum Taq polymerase (Invitrogen) according to the instructions of the supplier (annealing temperature of 55° C.). pHPLT contains the LAT promoter (P_{LAT}), a sequence encoding the LAT signal peptide (preLAT), followed by PstI and HpaI restriction sites for cloning. Variant alpha amylase (VAA) was created as PstI-HpaI fragment by fusion PCR (necessary to remove the internal PstI site in the EBS2 gene) with High Fidelity Platinum Taq Polymerase (Invitrogen, Carlsbad, Calif., USA) according to the instructions of the supplier and the following primers:

VAA(Pstl)_FW:

(SEQ ID NO:_{——})

gaatgtctacagcttcagcagccgcaccgtttaacggcaccatg
VAA(Hpal)_RV

(SEQ ID NO:_{——})

cccggggttaactcaaggccatgccaccaaccgtgg
VAAdelPstl_fw

(SEQ ID NO:_{——})

cccggccaagcgcttcagtcatgggtcgac
VAAdelPstl_rv

(SEQ ID NO:_{——})

gtcgacccatgactgaagcgcttggccggg

The PstI-HpaI fragment encoding variant alpha amylase was ligated with T4 DNA ligase according to the instructions of the supplier (Invitrogen) into PstI and HpaI digested pHPLT and transformed into B. subtilis strain OS14. The sequence of the LAT-VAA gene fusion was confirmed by DNA sequencing (BaseClear, Leiden, The Netherlands) and one of the correct plasmid clones was designated pHPLT-VAAc1 (FIG. 8). This plasmid was introduced into an amylase negative B. licheniformis host (BML612) by protoplast transformation (Pragai et al., Microbiology (1994) 140:305-310). Neomycin resistant transformants secrete variant alpha amylase as judged by halo formation on starch plates after iodine staining (FIG. 9).

Next plasmid plCatH-VAAc1 (ori2) (FIG. 10) was created by inserting the LAT-VAA gene construct from pHPLT-VAAc1 into the vector plCatH. plCatH contains the following features: an origin of replication (ori pE194, for replication in Bacillus [Horinouchi, S, et al, J. Bacteriol. 150(2):804-14 (1982)]), ori pBR322 (for amplification in E. coli), a neomycin resistance gene for selection, and the native B. licheniformis chloramphenicol resistance gene (cat) for selection, chromosomal integration and cassette amplification. The preLAT-precursorVAA gene fusion (including the LAT promoter and the LAT transcription terminator) was amplified form pHPLT-VAAc1 with the primers:

(SEQ ID NO:_{——})

| | |
|---|---|
| VAAXhol_FW | atcctactcgaggcttttcttttggaagaaaatataggg |

(SEQ ID NO:_{——})

| | |
|---|---|
| VAAXhol_RV | tggaatctcgaggttttatcctttaccttgtctcc |

The resulting PCR fragment was digested with XhoI, ligated into XhoI digested plCatH and transformed into B. subtilis strain OS14 as described in U.S. Patent Application US20020182734 (International Publication WO 02/14490). Plasmid DNA was isolated from an amylase positive transformant and the sequence of the VAA gene construct was confirmed by DNA sequencing. The plasmid of one correct clone was designated plCatH-VAA2c1 (ori2) and then transformed into B. licheniformis strain BML612 (BRA7 derivative, cat-, amyL-, spo-) at the permissive temperature (37° C.). One amylase positive, neomycin resistant (neoR) and chloramphenicol resistant (CmR) transformant was selected and designated BML612(plCatH-VMc1). The plasmid in BML612(plCatH-VAAc1) was integrated into the cat region on the B. licheniformis genome by growing the strain at a non-permissive temperature (50° C.) in medium with 5 μg/ml chloramphenicol. One CmR resistant clone was selected and designated BML612-plCatH-VAAc1. BML612-plCatH-VAAc1 was grown again at the permissive temperature for several generations without antibiotics to loop-out vector sequences and then one neomycin sensitive (neoS), CmR clone was selected. In this clone, vector sequences of plCatH on the chromosome are excised (including the neomycin resistance gene) and only the VAA-cat cassette is left. Next, the VAA-cat cassette on the chromosome was amplified by growing the strain in/on media with increasing concentrations of chloramphenicol. After various rounds of amplification, one clone (resistant against 75 μg/ml chloramphenicol) was selected and designated BML612-VAAc1.

### Example 2

### Assay For Determining α-Amylase Activity

Soluble Substrate Assay: A rate assay was developed based on an end-point assay kit supplied by Megazyme (Aust.) Pty. Ltd. A vial of substrate (p-nitrophenyl maltoheptaoside, BPNPG7) was dissolved in 10 ml of sterile water followed by a 1:4 dilution in assay buffer (50 mM maleate buffer, pH 6.7, 5 mM calcium chloride, 0.002% Tween20). Assays were performed by adding 10 μl of amylase to 790 μl of the substrate in a cuvette at 25° C. Rates of hydrolysis were measured as the rate of change of absorbance at 410 nm, after a delay of 75 seconds. The assay was linear up to rates of 0.2 absorption units/min.

α-Amylase protein concentration was measured using the standard Bio-Rad Assay (Bio-Rad Laboratories) based on the method of Bradford, Anal. Biochem., Vol. 72, p. 248 (1976) using bovine serum albumin standards.

### Example 3

### Preparation and Testing of Additional Mutant Alpha-Amylases for Thermal Stability

Variant B. stearothermophilus alpha-amylase was prepared having deletions at R179/G180 as described in Example 1. Thermal inactivation rate for the mutant will be measured according to the following procedure. Amylase stock solutions will be dialysed extensively into 20 mM ammonium acetate, 4 mM CaCl₂ pH 6.5. Each sample will be stored at 4° C. For measurement of stability, this stock will be diluted >50 fold into 50 mM ammonium acetate, 5 mM CaCl₂, 0.02% Tween 20 pH 4.8 to a final concentration of between 30 and 50 μg/ml. Six 100 μl aliquots will be put into eppendorf tubes and placed into a water bath or hot block at 83° C. The eppendorf tubes will be removed at regular, measured intervals of between 30 seconds and 5 minutes and placed on ice to stop the inactivation. The residual activity will be assayed using a soluble substrate as described in Example 2. The natural log of the activity was plotted against time of incubation, and the rate constant for inactivation obtained from the slope of the straight line. Results will be provided

It is anticipated that the mutant enzymes having introduced therein the mutations according to the invention will have significantly improved stability under the conditions of the assay.

The alpha amylase activity can be measured by a colorimietric method that monitors the rate of degradation of p-nitophenyl amltoheptoside. The rate of p-nitrophenyl release is proportional to amylase activity and is monitored at 410 nm.

### Example 3a

Schrool Method (Fehling's Assay) for Determining the DE of a Slurry

Reagent Solutions

Fehlings solutions A & B: (VWR, Brisane, Calif. Catalogue # VW3316-2; VW3317-1)

A Potassium Iodide (30% w/v) solution was prepared by dissolving 150 g Kl in 450 ml distilled water. 1.5 ml 1 N NaOH was added thereto. This solution was quantitatively transferred to a 500 ml volumetric flask and brought to the mark with distilled water.

A Sulfuric Acid (26% w/v) solution was prepared by gentle agitating, slowly adding 72.5 ml concentrated sulfuric acid (S.G. 1.84) to 400 ml distilled water in a 600 ml beaker. The solution was then cooled to room temperature. This solution was quantitatively transferred to a 500 ml volumetric flask and bring to the mark with distilled water.

A Starch Indicator solution was prepared as follows. 150 g of NaCl was dissolved in 300 ml distilled water and heated to boiling. A slurry of starch was prepared in cold distilled water containing 5 g (dry weight) of soluble starch. While agitating the hot NaCl solution, the NaCl solution was slowly added to the starch slurry. The resulting mixture was brought to a boil and boiled for 5 minutes, and then cooled to room temperature. The resulting solution was then quantitatively transfer to a 500 ml volumetric flask and bring to the mark with distilled water. Not all the salt will dissolve.

Glucose (1.00% w/v), standardized: glucose (Sigma-Aldrich, Saint Louis, Mo., USA)

Sodium Thiosulfate (VWR, International, Brisbane, Calif., Catalogue # EM-SX0810-11) (0.1N), standardized:

Assay Procedure

A heater was thoroughly warmed up and adjusted to bring 50 ml of water to a boil in 3 minutes. A sample of mash was obtained and a dilution was prepared containing the equivalent of 47 to 67 mg dextrose per 10 ml. For example, dilute about 15 g of liquefied mash (with DE=10-12) or 4 g of saccharified mash (with DE=50-60) to 100 ml. The % solids of the diluted solution was determined by refractometer measurements (Abbe Refractometer, Model 10450, American Optical Corporation—Scientific Instrument Division, Buffalo, N.Y., USA) using data tables (Corn Refiners Association, Washington, D.C., USA). 10 ml of diluted sample was transferred into a flask (250 ml Erlenmeyer flask (F is the weight of the flask)) and weighed (F+S). With mixing, 15 ml distilled water was added, then 10 ml Fehlings solution A, and 10 ml Fehlings solution B. The resulting mixture was brought to a boil on the heater in 3 minutes (+/−15 sec) and boiling continued for two more minutes. The resulting mixture was then cooled immediately under running tap water. To this mixture, 10 ml 30% Potassium Iodide and then 10 ml 26% Sulfuric Acid were added by mixing. 2 ml Starch Indicator were then added and mixed. The resulting mixture was titrated immediately with 0.1 N Sodium Thiosulfate until the blue starch-iodine complex disappears. The blue color should not reappear for at least one minute. The titration volume (TVs) was recorded. For a standard, 5.00 ml of 1.00% Glucose and 20 ml distilled water were transferred into a 250 ml Erlenmeyer flask. For a water blank, pipet 25 ml of distilled water into another flask. Return to the addition of Fehlings solution A and B step and follow through the procedure for each flask. The titration volumes were recorded (TVstd and TVwb, respectively).
Calculations

$$\% DE = \frac{5 \times ( TVwb - TVs ) \times 100}{\% S \times [ ( F + S ) - F ] \times ( TVwb - TVstd )}$$

### Example 4

### Determination of Average DE Progression

Alpha amylase [EBS2] produced as described in Example 1, was provided by Genencor international (Palo Alto, Calif.). 380 grams of corn starch (Archer Daniels Midland 106-B Pearl Corn Starch, Decatur, Ill., USA) were suspended in 1000 ml of water and the pH of the slurry was adjusted to pH 5.5. The slurry was stirred overnight for hydration of the starch (12 hours) and the pH was adjusted with 6.0 N H₂SO₄ until the pH was stabilized. 20 ppm Ca²⁺, 100 ppm SO₂, and the variant alpha amylase was added at 2 A-10 units/gram or 0.28 kg/MT dry solids and run through a jet cooker at 106.5° C. for 5 minutes cooled and then incubated at 95° C. for 120 minutes in a water bath. The dissolved solid (DS) was ascertained to be about 35 ds, using a Abbe refractometer (Model 10450, American Optical Corporation—Scientific Instrument Division, Buffalo, N.Y., USA) at 30° C. and using the tables provided by the Corn Refiners Association (Washington, D.C.) Critical Data Tables. The samples were withdrawn at 30 minute intervals and the DE was measured by Schrool Fehlings titration method (See Example 3a). The results are shown in FIG. 11.

### Example 5

### Slurry Viscosity Progression

The variant alpha amylase was produced as described in Example 1. 810 grams of ground corn were suspended in 2 liters of water containing 30% of the 2 liters as thin stillage within a water jacketed, glass jar device within a IKA EUROSTAR Labortechnik Power control—visc P7 viscometer with a Viscoklick VK12 controller. The pH of the slurry was adjusted to pH 5.5 with 6.0 N H₂SO₄. The slurry was stirred for a 30 minutes at room temperature and the temperature of the slurry was raised to 60 degrees C. and raised at 1 degree per minute to 85 degrees The variant alpha amylase was added immediately as the slurry was heating at 4 A-10 units/g. The viscosity was measured at 4 minute intervals.

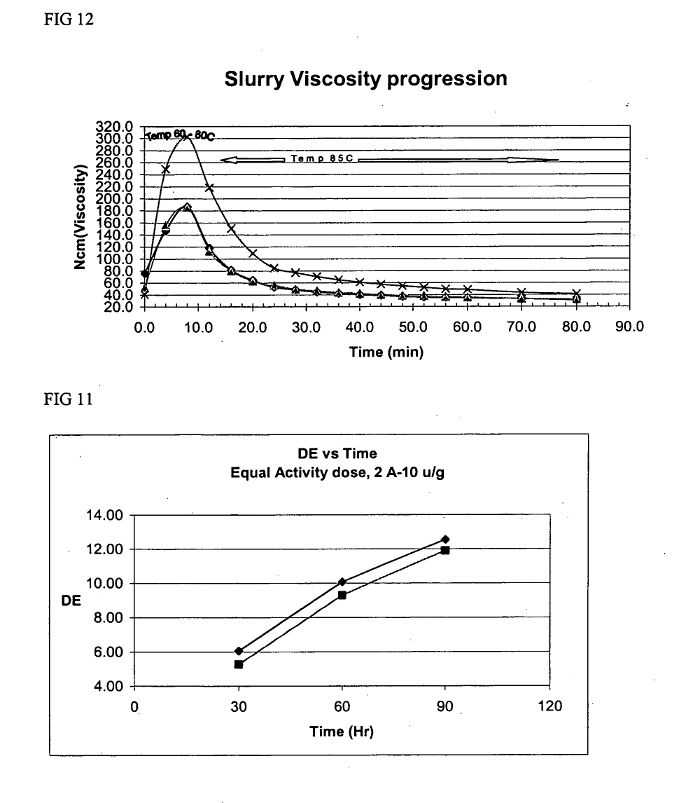

The results are shown in FIG. 12. Whereas when the variant licheniformis alpha amylase sold by GENENCOR has a peak of about 300 Ncm, the variant alpha amylase has a peak of about 185 Ncm

### Example 6

### LC/MS Analysis

The Geobacillus stearothermophilus α-amylase expressed in Example 1 was analyzed by LC/MS. All liquid samples were precipitated with 10% TCA followed by the reduction reactions with 20 mM DTT @ 50° C. for 15-20 min. The alkylation reaction was also performed with 55 mM iodoacetamide. Allow the alkylation reaction in dark for 45 min at room temp. Proteolytic digestions were performed by incubation with various proteases in 25 mM ammonium bicarbonate for 4 hr at 37° C. (enzyme to substrate ratio was 1:20).

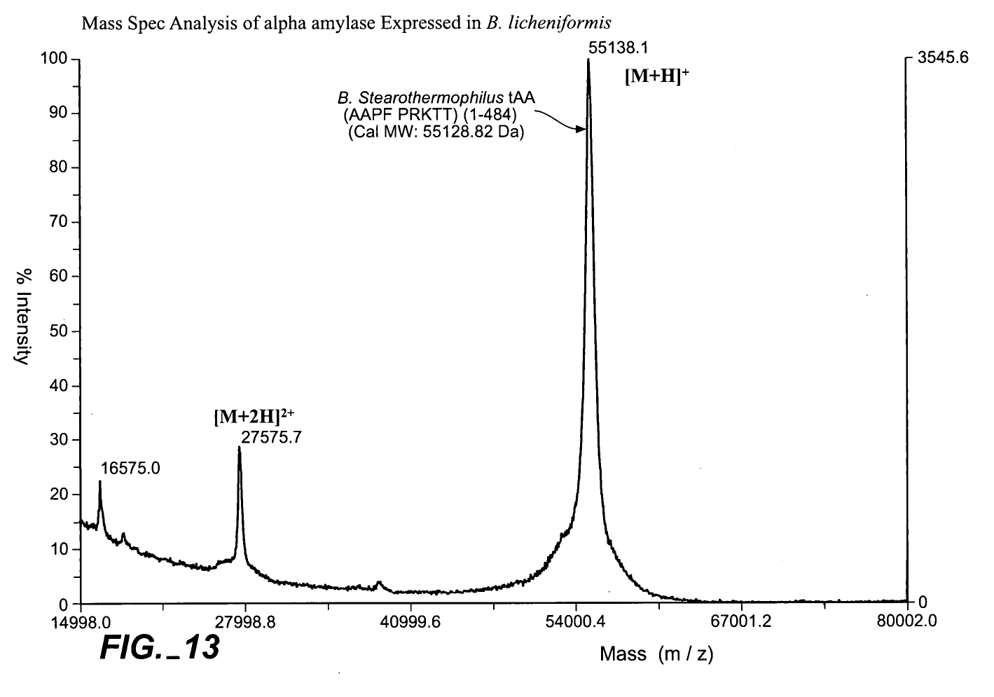

All MS and MS/MS data were acquired using the Surveyor HPLC system coupled to the LCQ Advantage Ion Trap MS (ThermoFinnigan, San Jose, Calif.). A Vydac reverse phase C18 column (2.1×150 mm) was used for all proteolytic digested samples using the HPLC gradient from 0% to 70% Solvent B over 65 minutes at the flow rate of 200 μL/min. Solvent A (0.1% TFA in water) and Solvent B (0.08% TFA in acetonitrile). Data Processing was performed using the TurboSEQUEST and the Xcalibur programs (ThermoFinnigan). The results are shown in FIGS. 13 and 14. LC/MS data from three proteolytic digestions (trypsin, chymotrypsin and Glu-C) confirmed approximately 83% of the protein sequence. The RG deletion in this protein was also confirmed. No C-terminal peptide with sequence (VSTIARPITTRPWTGEFVRWTEPRLVAWP [SEQ ID NO:17]) could be found.

What is claimed is:

1. A variant of a precursor α-amylase comprising deletions at one or more of the following positions R179 and G180 wherein the precursor α-amylase is selected from the group consisting of a Bacillus stearothermophilus α-amylase having an amino acid sequence shown in SEQ ID NO:3, an α-amylase which displays at least 90% identity with the amino acid sequence of SEQ ID NO:3, an α-amylase which displays at least 90% identity with the amino acid sequence of SEQ ID NO:2, and an α-amylase which has the amino acid sequence of SEQ ID NO:4.

2. The variant α-amylase according to claim 1 wherein the precursor α-amylase is a Bacillus stearothermophilus α-amylase having an amino acid sequence shown in SEQ ID NO.:3.

3. The variant α-amylase according to claim 1 wherein the precursor α-amylase is an α-amylase which displays at least 90% identity with the amino acid sequence of SEQ ID NO.:3.

4. The variant α-amylase according to claim 1 wherein the precursor α-amylase is an α-amylase which displays at least 90% identity with the amino acid sequence of SEQ ID NO:2.

5. The variant α-amylase according to claim 1 wherein the precursor α-amylase is an α-amylase which has the amino acid sequence of SEQ ID NO.:4.

6. A variant α-amylase selected from the group consisting of an α-amylase having the amino acid sequence of SEQ ID NO:16 and an α-amylase which has at least 97% identity with the amino acid sequence of SEQ ID NO:16.

7. The variant α-amylase according to claim 6 wherein the variant α-amylase has the amino acid sequence of SEQ ID NO:16.

8. The variant α-amylase according to claim 6 wherein the variant α-amylase has at least 97% identity with the amino acid sequence of SEQ ID NO:16.

9. A DNA encoding the variant α-amylase according to claim 1.

10. An expression vector comprising the DNA according to claim 9.

11. A host cell transformed with the expression vector according to claim 10.

12. The host cell according to claim 11 wherein the host cell is a Bacillus sp.

13. The host cell according to claim 12 wherein the host cell is selected from the group of Bacillus subtilis and Bacillus licheniformis.

14. A detergent composition comprising the variant α-amylase according to claim 1.

15. A starch liquefying composition comprising the variant α-amylase according to claim 1.

16. A method of liquefying starch comprising the steps of contacting a starch slurry with a variant α-amylase according to claim 1, raising the temperature of the slurry to between 60 to 80° C.; and maintaining the viscosity of the slurry below 200.0 Ncm

17. A method of liquefying starch comprising the steps of contacting a starch slurry with a variant α-amylase according to claim 1, raising the temperature of the slurry to between 85 to 100° C.; and providing an average DE progression of at least 8.00 within 60 minutes of the onset of secondary liquefaction.

18. A method of producing a variant α-amylase having amylolytic activity comprising, a) stably transforming a host cell with an expression vector according to claim 10; b) cultivating the transformed host cell under conditions suitable for said host cell to produce an enzyme having amylolytic activity; and c) recovering said variant α-amylase.
